SEQUENCE LISTING (82 sequences)

SEQ ID NO: 1 - RNA, 976 nt, Hepatitis C <s300> Takamizawa A, Mori C, Fuke I et al. Structure and organization of the hepatitis C virus genome isolated from human carriers Journal of Virology 65 1105-1113 1991
cgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccaaccgccgcccacaggacgucaaguucccgggcgguggucagaucguugguggaguuuaccuguugccgcgcaggggccccagguugggugugcgcgcgcccaggaagacuuccgagcggucgcaaccucguggaaggcgacaaccuauccccaaggcucgccggcccgagggcaggaccugggcucagcccggguacccuuggccucucuauggcaaugaaggcuuagggugggcaggauggcuccugucaccccgcggcucccggccuaguuggggccccacggacccccggcguaggucgcguaauuuggguaaggucaucgauacccucacaugcggcuucgccgaucucaugggguacauuccgcucgucggcgccccccuggggggcgcugccagggcccuggcacaugguguccggguucuggaggacggcgugaacuaugcaacagggaaucugcccgguugcucuuuuucuaucuuccucuuggcucugcuguccugccugaccaccccagcuuccgcuuacgaagugcacaacguguccgggauauaucaugucacgaacgacugcuccaacgcaagcauuguguauga

SEQ ID NO: 2 - DNA, 13 bp, Artificial sequence
  Synthetic Type I catalytic domain
ccgagccggacga

SEQ ID NO: 3 - DNA, 15 bp, Artificial sequence
  Synthetic; Type II catalytic domain
ggctagctacaacga

SEQ ID NO: 4 - DNA, 23 bp, Artificial sequence
  Synthetic; sense primer encoding bacteriophase T7 RNA Polymerase
tgtaatacgactcactatagcga

SEQ ID NO: 5 - DNA, 21 bp, Artificial sequence
  Synthetic; antisense HCV primer
tcatacacaatgcttgcgttg

SEQ ID NO: 6 - DNA, 30 bp, Artificial sequence
  Synthetic; sense primer comprising HindII restriction site
cccaagcttgggtgaggaactactgtcttc

SEQ ID NO: 7 - DNA, 30 bp, Artificial sequence
  Synthetic; antisense primer containing a NotI restriction site
ttaagcggccgcaaatctgcctcatacaca

SEQ ID NO: 8 - DNA, 24 bp, Artificial sequence
  Synthetic; HCV-derived sense primer
aaggccttgtggtactgcctgata

SEQ ID NO: 9 - DNA, 28 bp, Artificial sequence
  Synthetic; HCV-derived 6′-carboxyfluorescein succinimidyl ester (FAM)-labeled probe
accgtgcaccatgagcacgaatcctaaa

SEQ ID NO: 10 - DNA, 24 bp, Artificial sequence
  Synthetic; antisense HCV-derived primer
ggcggttggtgttacgtttggttt

SEQ ID NO: 11 - DNA, 24 bp, Artificial sequence
  Synthetic; sense primer for neomycin resistance gene
accttgctcctgccgagaaagtat

SEQ ID NO: 12 - DNA, 25 bp, Artificial sequence
  Synthetic; 5′ cyanine-5 labeled probe for neomycin resistance gene
aatgcggcggctgcatacgcttgat

SEQ ID NO: 13 - DNA, 24 bp, Artificial sequence
  Synthetic; antisense primer for neomycin resistance gene
cgatgtttcgcttggtggtcgaat

SEQ ID NO: 14 - DNA, 9 bp, Artificial sequence
  Synthetic; 5′ recognition binding arm
tctttgagg

SEQ ID NO: 15 - DNA, 15 bp, Artificial sequence
  Synthetic; 3′ recognition binding arm
ttaggattcgtgctc

SEQ ID NO: 16 - RNA, 25 nt, Artificial sequence
  Synthetic; HCV RNA target
gagcacgaauccuaaaccucaaaga

SEQ ID NO: 17 - DNA, 9 bp, Artificial sequence
  Synthetic; 5′ recognition binding arm
gggtatcga

SEQ ID NO: 18 - DNA, 15 bp, Artificial sequence
  Synthetic; 3′ recognition binding arm
gaccttacccaaatt

SEQ ID NO: 19 - RNA, 24 nt, Artificial sequence
  Synthetic; HCV RNA target
aauuuggguaaggucaucgauacc

SEQ ID NO: 20 - DNA, 9 bp, Artificial sequence
  Synthetic; 5′ recogntion binding arm
agaggaaga

SEQ ID NO: 21 - DNA, 15 bp, Artificial sequence
  Synthetic; 3′ recognition binding arm
agaaaaagagcaacc

SEQ ID NO: 22 - RNA, 25 nt, Artificial sequence
  Synthetic; HCV RNA target
gguugcucuuuuucuaucuuccucu

SEQ ID NO: 23 - DNA, 15 bp, Artificial sequence
  Synthetic; 5′ recognition binding arm
gagccaagaggaaga

SEQ ID NO: 24 - DNA, 15 bp, Artificial sequence
  Synthetic; 3′ recognition binding arm
agaaaaagagcaacc

SEQ ID NO: 25 - RNA, 32 nt, Artificial sequence
  Synthetic; HCV RNA target
gguugcucuuuuucuaucuuccucuuggcucu

SEQ ID NO: 26 - DNA, 15 bp, Artificial sequence
  Synthetic; mutated type II catalytic domain
ggcgagctacaacga

SEQ ID NO: 27 - DNA, 39 bp, Artificial sequence
  Synthetic; Dz348-9-15
tctttgaggggctagctacaacgattaggattcgtgctc

SEQ ID NO: 28 - DNA, 39 bp, Artificial sequence
  Synthetic; Dz348mut-9-15
tctttgaggggcgagctacaacgattaggattcgtgctc

SEQ ID NO: 29 - DNA, 45 bp, Artificial sequence
  Synthetic; Dz348-15-15
ggtttttctttgaggggctagctacaacgattaggattcgtgctc

SEQ ID NO: 30 - DNA, 39 bp, Artificial sequence; identical to SEQ ID NO: 27
  Synthetic; Dz348-9-15end

SEQ ID NO: 31 - DNA, 39 bp, Artificial sequence; identical to SEQ ID NO: 28
  Synthetic; Dz348mut-9-15-end

SEQ ID NO: 32 - DNA, 45 bp, Artificial sequence; identical to SEQ ID NO: 29
  Synthetic; Dz348-15-15end

SEQ ID NO: 33 - DNA, 33 bp, Artificial sequence
  Synthetic; Dz348-10-8end
ttctttgaggggctagctacaacgattaggatt

SEQ ID NO: 34 - DNA, 39 bp, Artificial sequence
  Synthetic; Dz699-9-15
gggtatcgaggctagctacaacgagaccttacccaaatt

SEQ ID NO: 35 - DNA, 39 bp, Artificial sequence
  Synthetic; Dz699mut-9-15
gggtatcgaggcgagctacaacgagaccttacccaaatt

SEQ ID NO: 36 - DNA, 42 bp, Artificial sequence
  Synthetic; Dz699-12-15
tcagggtatcgaggctagctacaacgagaccttacccaaatt

SEQ ID NO: 37 - DNA, 45 bp, Artificial sequence
  Synthetic; Dz699-15-15
atgtcagggtatcgaggctagctacaacgagaccttacccaaatt

SEQ ID NO: 38 - DNA, 39 bp, Artificial sequence; identical to SEQ ID NO: 34
  Synthetic; Dz699-9-15end

SEQ ID NO: 39 - DNA, 39 bp, Artificial sequence; identical to SEQ ID NO: 34
  Synthetic; Dz699mut-9-15

SEQ ID NO: 40 - DNA, 11 bp, Artificial sequence
  Synthetic; sequence comprising HindIII site of sense primer SEQ ID NO.6
cccaagcttgg

SEQ ID NO: 41 - DNA, 17 bp, Artificial sequence
  Synthetic; sequence comprising NotI site of antisense primer SEQ ID NO.7
ttaagcggccgcaaatc

SEQ ID NO: 42 - DNA, 45 bp, Artificial sequence; identical to SEQ ID NO: 37
  Synthetic; Dz699-15-15end

SEQ ID NO: 43 - DNA, 45 bp, Artificial sequence; identical to SEQ ID NO: 37
  Synthetic; Dz699mut-15-15end

SEQ ID NO: 44 - DNA, 39 bp, Artificial sequence
  Synthetic; Dz858-9-15
agaggaagaggctagctacaacgaagaaaaagagcaacc

SEQ ID NO: 45 - DNA, 39 bp, Artificial sequence
  Synthetic; Dz858mut-9-15
agaggaagaggcgagctacaacgaagaaaaagagcaacc

SEQ ID NO: 46 - DNA, 42 bp, Artificial sequence
  Synthetic; Dz858-12-15
ccaagaggaagaggctagctacaacgaagaaaaagagcaacc

SEQ ID NO: 47 - DNA, 45 bp, Artificial sequence
  Synthetic; Dz858-15-15
gagccaagaggaagaggctagctacaacgaagaaaaagagcaacc

SEQ ID NO: 48 - DNA, 39 bp, Artificial sequence; identical to SEQ ID NO: 44
  Synthetic; Dz858-9-15end

SEQ ID NO: 49 - DNA, 39 bp, Artificial sequence; identical to SEQ ID NO: 45
  Synthetic; Dz858mut-9-15end

SEQ ID NO: 50 - DNA, 45 bp, Artificial sequence; identical to SEQ ID NO: 47
  Synthetic; Dz858-15-15end

SEQ ID NO: 51 - DNA, 45 bp, Artificial sequence; identical to SEQ ID NO: 47
  Synthetic; Dz858mut-15-15end

SEQ ID NO: 52 - DNA, 45 bp, Artificial sequence; identical to SEQ ID NO: 47
  Synthetic; Dz858-15-15 2Mend

SEQ ID NO: 53 - DNA, 45 bp, Artificial sequence; identical to SEQ ID NO: 47
  Synthetic; Dz858-15-15 4Mend

SEQ ID NO: 54 - RNA, 17 nt, Artificial sequence
  Synthetic; HCV target
cuuucucuaucuuccuc

SEQ ID NO: 55 - DNA, 45 bp, Artificial sequence; identical to SEQ ID NO: 47
  Synthetic; DZ858-15-15 end generic

SEQ ID NO: 56 - DNA, 45 bp, Artificial sequence
  Synthetic; Dz858-15-15 4Me6Mc
gagccaagaggaagaggctagcnacaacgaagaaaaagagcaacc

SEQ ID NO: 57 - DNA, 45 bp, Artificial sequence; identical to SEQ ID NO: 56
  Synthetic; Dz858-15-15 6Mc

SEQ ID NO: 58 - DNA, 24 bp, Artificial sequence
  Synthetic; HCV sense primer
ggcgtgaactatgcaacagggaat

SEQ ID NO: 59 - DNA, 26 bp, Artificial sequence
  Synthetic; 6′-carboxyfluorescein, succinimidyl ester (FAM) labeled HCV probe
ttccgcttacgaagtgcacaacgtgt

SEQ ID NO: 60 - DNA, 24 bp, Artificial sequence
  Synthetic; HCV antisense primer
tggagcagtcgttcgtgacatgat

SEQ ID NO: 61 - DNA, 25 bp, Artificial sequence
  Synthetic; 6′-carboxy-2′-4,4′,5′,7, 7′-hexachlorofluorescein,succinimidyl ester (HEX)-labeled neo probe
aatgcggcggctgcatacgcttgat

SEQ ID NO: 62 - DNA, 45 bp, Artificial sequence
  Synthetic; Dz858-15-15 morpholino variant
gagccaagaggaagaggctagctacaacgaaganaaagagcaacc

SEQ ID NO: 63 - DNA, 45 bp, Artificial sequence
  Synthetic; Dz858mut-15-15 morpholino variant
gagccaagaggaagaggcgagctacaacgaaganaaagagcaacc

SEQ ID NO: 64 - DNA, 20 bp, Artificial sequence
  Synthetic; human GAPDH sense primer
tccctcaagattgtcagcaa

SEQ ID NO: 65 - DNA, 20 bp, Artificial sequence
  Synthetic; human GAPDH antisense primer
agatccacaacggatacatt

SEQ ID NO: 66 - DNA, 31 bp, Artificial sequence
  Synthetic; DNAzyme
gacgaagaggctagctacaacgaagagaaag

SEQ ID NO: 67 - DNA, 31 bp, Artificial sequence
  Synthetic; DNAzyme
gtttaggaggctagctacaacgatcgtgctc

SEQ ID NO: 68 - DNA, 31 bp, Artificial sequence
  Synthetic; DNAzyme
tcaccttaggctagctacaacgaccaagtta

SEQ ID NO: 69 - DNA, 39 bp, Artificial sequence; identical to SEQ ID NO: 44
  Synthetic; Dz858-9-15end generic

SEQ ID NO: 70 - DNA, 39 bp, Artificial sequence; identical to SEQ ID NO: 44
  Synthetic; Dz858-9-15- O-methyl generic

SEQ ID NO: 71 - DNA, 45 bp, Artificial sequence; identical to SEQ ID NO: 62
  Synthetic; Dz858-15-15 generic

SEQ ID NO: 72 - DNA, 45 bp, Artificial sequence; identical to SEQ ID NO: 47
  Synthetic; Dz858-15-15 O-methyl generic

SEQ ID NO: 73 - DNA, 39 bp, Artificial sequence
  Synthetic; DZ858-9-15 generic
agaggaagaggctagctacaacgaaganaaagagcaacc

SEQ ID NO: 74 - DNA, 39 bp, Artificial sequence; identical to SEQ ID NO: 27
  Synthetic; Dz348-9-15 generic

SEQ ID NO: 75 - DNA, 45 bp, Artificial sequence; identical to SEQ ID NO: 29
  Synthetic; Dz348-15-15 generic

SEQ ID NO: 76 - DNA, 39 bp, Artificial sequence; identical to SEQ ID NO: 34
  Synthetic; Dz699-9-15 generic

SEQ ID NO: 77 - DNA, 45 bp, Artificial sequence; identical to SEQ ID NO: 37
  Synthetic; Dz699-15-15 generic

SEQ ID NO: 78 - DNA, 45 bp, Artificial sequence
  Synthetic; mtDz858Sen
ggttgctctttttctggcgagctacaacgatcttcctcttggctc

SEQ ID NO: 79 - DNA, 45 bp, Artificial sequence
  Synthetic; Dz2
gcacggtctacgagaggctagctacaacgactcccggggcactcg

SEQ ID NO: 80 - DNA, 45 bp, Artificial sequence; identical to SEQ ID NO: 79
  Synthetic; Dz2-4-Ome

SEQ ID NO: 81 - DNA, 21 bp, Artificial sequence
  Synthetic; b-actin sense primer
ccttcctgggcatggagtcct

SEQ ID NO: 82 - DNA, 21 bp, Artificial sequence
  Synthetic; b-actin antisense primer
ggagcaatgatcttgatcttc

(19) United States
(12) Patent Application Publication
(10) Pub. No.: US 2009/0203890 A1
(43) Pub. Date: Aug. 13, 2009

(54) HEPATITIS C ANTIVIRALS

(75) Inventors: Carolina Alfieri, Dollard-des-Ormeaux (CA); Janie Trepanier, New York, NY (US); Jerome E. Tanner, Dollard-des-Ormeaux (CA); Richard Momparler, Outremont (CA)

Correspondence Address: CHOATE, HALL & STEWART LLP, TWO INTERNATIONAL PLACE, BOSTON, MA, 02110, US

(73) Assignee: Chu Sainte-Justine, Le Centre Hospitalier Universitaire Mere-Enfant, Montreal (CA)

(21) Appl. No.: 12/208,282

(22) Filed: Sep. 10, 2008

Related U.S. Application Data
(63) Continuation-in-part of application No. 11/997,548.
(60) Provisional application No. 60/703,879, filed on Aug. 1, 2005.

(51) Int. Cl. A61K 31/7088; C07H 21/04; A61P 31/12

(52) U.S. Cl. 536/23.1

(57) ABSTRACT

The present invention relates to deoxyribozymes targeting and cleaving HCV RNA. More particularly, the present invention relates to deoxyribozymes and composition used for the inhibition of HCV replication and HCV-related diseases.

### CROSS-REFERENCE TO RELATED APPLICATION

This application is a continuation-in-part of U.S. patent application Ser. No. 11/997,548 filed on Aug. 1^{st}, 2006, which claims the benefit of priority of U.S. Provisional Patent Application No. 60/703,879 filed on Aug. 1^{st}, 2005, the disclosures of which are incorporated herein by reference.

### FIELD OF THE INVENTION

The present invention relates to deoxyribozymes targeting and cleaving HCV RNA. More particularly, the present invention relates to deoxyribozymes and composition used for the inhibition of HCV replication.

### BACKGROUND OF THE INVENTION

Hepatitis C virus (HCV) infection represents an important global health problem. HCV infection is a major cause of chronic liver disease, a condition which, if left untreated, can eventually lead to hepatocellular carcinoma or outright liver failure [1]. HCV is a single-stranded positive RNA virus which replicates through a viral RNA-dependent RNA polymerase. The replication cycle of HCV thus involves a step of conversion of the positive RNA strand into a negative RNA strand.

Current antiviral therapeutics for HCV has proven inadequate in stemming the disease process. HCV therapy for acute and chronic HCV infection consists of a combination of interferon-α and the nucleoside analog, ribavirin [2]. In spite of the encouraging results obtained with this combination therapy, over 50% of treated patients fail to achieve a stable virus load or virus clearance [3]. Given the current lack of an effective vaccine [35] and an increasing risk of drug resistance due to HCV's high rate of mutation, pursuit of alternative HCV therapeutics remains a pressing issue [6, 36, 37]. While various therapeutic stratagems for HCV are undergoing clinical testing and include drugs which inhibit virus protein processing or virus RNA replication [3, 4], many of these agents will likely lose therapeutic effectiveness due to HCV's high rate of mutation and ensuing drug resistance [5]. Thus the development of alternative HCV therapeutics will remain a pressing issue for the foreseeable future [6].

One strategy currently under intense investigation is concerned with attempts to cleave HCV genomic RNA with either ribozymes or deoxyribozymes [7, 8]. RNAzymes, also referred to as ribozymes, were originally discovered in plants as self-cleaving motifs encoded within the genome of a number of small, circular pathogenic RNA viruses [9]. Subsequently, RNAzymes have been genetically modified to recognize and cut aberrant cellular mRNAs or the RNA genomes of certain human viruses [8, 10]. Unfortunately RNAzymes suffer the disadvantages of a short half-life due to biological instability, difficulty in large-scale synthesis and a possible loss in biologic activity when encountering RNAs with alternative base substitutions [11].

A novel therapeutic strategy involves the use of deoxyribozymes, also known as DNA enzymes or DNAzymes. Deoxyribozymes have been shown in several animal models to reduce the expression of detrimental RNAs and to abrogate disease pathology [44-46]. Deoxyribozymes are currently in preclinical development for the treatment of cancer, genetic diseases and viral infection [7, 16-20]. For example, deoxyribozymes have been shown to cleave HIV-1 viral RNA in vitro and in vivo [18, 28, 29]. Therefore, these catalytic DNA molecules, designed to target and cleave specific RNA sequences, are promising for the treatment of various diseases. Deoxyribozymes were originally generated through a combination of chemical synthesis and high-throughput selection [15]. Deoxyribozymes are classified as type I or type II based on their catalytic domain nucleotide structure and their RNA target recognition sequence [15]. Type I deoxyribozymes contain a 13-base catalytic domain and cleave AA/G motifs, whereas type II deoxyribozymes have a catalytic domain nucleotide length of 15 bases and cleave AC/U or GC/U motifs.

Deoxyribozymes, by contrast to other nucleotide-based technologies, represent a more attractive HCV drug candidate due to their small size (30 to 40 bases or even higher e.g., 45, 50), ease of synthesis, and increased resistance to chemical or nuclease degradation [12]. Additionally, deoxyribozymes are enzymatically more efficient compared to RNAzymes, display greater target specificity and appear less demanding in their RNA target requirements [13, 14].

Deoxyribozymes are therefore rapidly moving from being a research laboratory tool to becoming a full-fledged pharmacological strategy for the treatment of various human diseases [10, 12].

Oketani et al., [7] describe DNAzymes targeting the non-coding region of HCV. Although Oketani describes efficient cleavage of the target HCV in cell-free assays, intracellular cleavage of HCV is only inferred from heterologous gene expression (e.g., luciferase). The intracellular or in vivo effect of Oketani's DNAzymes on HCV genome cleavage has not been shown.

A recognized problem with DNAzymes, is that although they may cleave their target efficiently in vitro, their activity or efficiency in cells expressing the target sequence, may be impaired. To our knowledge, none of the HCV DNAzyme developed to date has been shown to be efficiently cleaving their target in mammals.

For example, U.S. patent application Ser. No. 09/817,879 to Blatt et al., published under No. 2003/0171311 on Sep. 11, 2003 described several enzymatic DNA molecules targeting the HCV genome, irrespective of the accessibility of the target site. Among those enzymatic DNA molecules, Blatt describes rather short DNAzymes (covering about 17 bases of HCV) modified with an inverted deoxyabasic group at their 3′-end. However, Blatt does not describe DNAzymes which are efficient in mammalian cells, nor in mammals.

In an attempt to develop new HCV therapeutics, we designed and characterized deoxyribozymes that recognized and efficiently cleaved a highly conserved HCV genome sequence encoding the viral core protein. We have demonstrated herein that this technology may be promising as a therapeutic for HCV and may serve as an alternative or adjunct to current HCV drug therapy.

These deoxyribozymes showed significant cleavage activity against the HCV core protein target RNA in mammalian (e.g., human) cells and in mammals, and may therefore have potential as a therapeutic candidate for clinical trial in HCV infected patients. DNAzymes are designed to target not only the HCV genome (positive RNA strand) but also its replication intermediate (negative RNA).

### SUMMARY OF INVENTION

The present invention relates to deoxyribozymes targeting and cleaving HCV RNA. More particularly, the present invention relates to deoxyribozymes and composition used for the inhibition of HCV replication or for lowering HCV replication.

U.S. Pat. Nos. 5,807,718 and 6,326,174 describe enzymatic DNA molecules comprising a catalytic domain. Some of these catalytic domains may be used to generate variants of the deoxyribozymes of the present invention.

U.S. Pat. No. 6,110,642 describes enzymatic DNA molecules that contain modified nucleotides. U.S. Pat. No. 6,673,611 describes deoxyribozymes with novel chemical compositions. Some of these modified catalytic domains may be used to generate variants of the deoxyribozymes of the present invention.

Schubbert, S et al. describes deoxyribozymes comprising 2′-O-methyl modified catalytic core. Some of these modified catalytic domains may be used to generate variants of the deoxyribozymes of the present invention. For example, suitable catalytic domains described herein may be used in association with the first and second annealing arms described herein.

In a first aspect, the present invention provides a deoxyribozyme which may comprise a first and second annealing arm and may also comprise a catalytic region between the first and second annealing arm.

In accordance with the present invention, the first and second annealing arm may be substantially complementary to a target HCV core region (core-encoding region).

Further in accordance with the present invention, the catalytic region may enable the cleavage of the target HCV core region (core-encoding region). The target HCV core region may be located within a HCV genome and/or within a HCV transcriptome.

Also in accordance with the present invention, the HCV core-encoding region may be substantially conserved among HCV subtypes. It is to be understood herein that the HCV subtypes include those which may be found in Gene Bank. A representative HCV subtype may be found in Gene Bank under accession no. M58335.

A target HCV core-encoding region may be one which is accessible for annealing (hybridization) (e.g., substantially free of secondary structure), more particularly, a target which is accessible for annealing with a deoxyribozyme. In accordance with an embodiment of the present invention a core region of choice may be one which is near a loop or located on a loop.

The HCV core-encoding region may be located, for example, between nucleotide 1 and 976 (SEQ ID NO.:1) with reference to Gene Bank accession no. M58335.

In accordance with an embodiment of the present invention, the first and second annealing arm of the deoxyribozyme may each independently have from about 7 to 20 deoxyribonucleotides and the deoxyribozyme may bind for example, a HCV region located between nucleotide 330 and nucleotide 370 of the HCV sequence depicted in SEQ ID NO.:1.

In accordance with an embodiment of the present invention, the deoxyribozyme may be able to cleave the HCV region at a site defined by 5′-A₁-R/Y-A₂-3′, where A₁ is a first annealing region of, for example, about 7 to 20 nucleotides, A₂ is a second annealing region of, for example, about 7 to 20 nucleotides, where R may be A or G and where Y may be U or C.

The formula 5′-A₁-R/Y-A₂-3′ may represent consecutives nucleotides of a desired HCV region.

In accordance with the present invention R may be A and Y may be U or C.

Further in accordance with the present invention, the first and second annealing arm may each independently have from about 7 to 18 deoxyribonucleotides and the deoxyribozyme may bind a HCV region located between nucleotide 330 and nucleotide 370 of the HCV sequence depicted in SEQ ID NO.:1, or between nucleotide 330 and 365 of SEQ ID NO.:1, or between nucleotide 330 and 360 of SEQ ID NO.:1, or between nucleotide 335 and 360 of SEQ ID NO.:1.

Also in accordance with the present invention, the first and second annealing arm may each independently have from about 9 to 15 deoxyribonucleotides and the desired deoxyribozyme may bind a HCV region located, for example, between nucleotide 330 and nucleotide 370 of HCV sequence depicted in SEQ ID NO.:1 (e.g., or between nucleotide 330 and 365 of SEQ ID NO.:1, or between nucleotide 330 and 360 of SEQ ID NO.:1, or between nucleotide 335 and 360 of SEQ ID NO.:1).

In accordance with a particular embodiment of the present invention, the first and second annealing arms may be totally (100%) complementary to the HCV region depicted in SEQ ID NO.:1.

In accordance with an exemplary embodiment of the present invention the DNAzyme may comprise SEQ ID NO.:74 or SEQ ID NO.:75. In accordance with a further exemplary embodiment of the present invention the DNAzyme may consist in SEQ ID NO.:74 or SEQ ID NO.:75.

Also in accordance with a particular embodiment of the present invention, a sequence comprising or consisting of SEQ ID NO.:74 or SEQ ID NO.:75 may have (in the first and/or second annealing arm) at least one nucleotide which is not complementary to SEQ ID NO.:1. More particularly, a sequence comprising or consisting of SEQ ID NO.:74 or SEQ ID NO.:75 may have one nucleotide which is not complementary to SEQ ID NO.:1 in either one of the first or second annealing arm).

Further in accordance with the present invention, a sequence comprising or consisting of SEQ ID NO.:74 or SEQ ID NO.:75 may have other nucleotides or base which are modified.

In accordance with another particular embodiment of the present invention, the first or second annealing arm may possess one, two or three nucleotides which are not complementary to the HCV region depicted in SEQ ID NO.:1.

In accordance with an additional embodiment of the present invention, the first and second annealing arm may each independently have from about 7 to 20 deoxyribonucleotides and the desired deoxyribozyme may bind a HCV region located, for example, between nucleotide 676 and nucleotide 715 of HCV sequence depicted in SEQ ID NO.:1.

The first and second annealing arm may each independently have from about 7 to 18 deoxyribonucleotides and the deoxyribozyme may bind a HCV region located between nucleotide 676 and nucleotide 715 of HCV sequence depicted in SEQ ID NO.:1, or between nucleotide 678 and 712 of SEQ ID NO.:1, or between nucleotide 680 and 710 of SEQ ID NO.:1 or between nucleotide 684 and 708 of SEQ ID NO.:1.

Also in accordance with the present invention, the first and second annealing arm may each independently have from about 9 to 15 deoxyribonucleotides.

Again in accordance with the present invention, the first and second annealing arms may be totally (100%) complementary to the HCV region or may possess one, two or three nucleotides which are not complementary to the HCV region.

In accordance with an exemplary embodiment of the present invention the DNAzyme may comprise SEQ ID NO.:76 or SEQ ID NO.:77. In accordance with a further exemplary embodiment of the present invention the DNAzyme may consist in SEQ ID NO.:76 or SEQ ID NO.:77.

Also in accordance with a particular embodiment of the present invention, a sequence comprising or consisting of SEQ ID NO.:76 or SEQ ID NO.:77 may have (in the first and/or second annealing arm) at least one nucleotide which is not complementary to SEQ ID NO.:1. More particularly, a sequence comprising or consisting of SEQ ID NO.:76 or SEQ ID NO.:77 may have one nucleotide which is not complementary to SEQ ID NO.:1 in either one of the first or second annealing arm).

Further in accordance with the present invention, a sequence comprising or consisting of SEQ ID NO.:76 or SEQ ID NO.:77 may have other nucleotides or base which are modified.

In accordance with a further embodiment of the present invention, the first and second annealing arm of the deoxyribozyme may each independently have from about 7 to 20 deoxyribonucleotides and the desired deoxyribozyme may bind a HCV region located between nucleotide 835 and nucleotide 880 of HCV sequence depicted in SEQ ID NO.:1.

In accordance with the present invention, the first and second annealing arm of the deoxyribozyme may each independently have from about 7 to 18 deoxyribonucleotides and the resulting deoxyribozyme may bind a HCV region located between nucleotide 835 and nucleotide 880 of HCV sequence depicted in SEQ ID NO.:1, or between nucleotide 838 and 878 of SEQ ID NO.:1 or between nucleotide 840 and 875 of SEQ ID NO.:1, or between nucleotide 842 and 874 of SEQ ID NO.:1 or between 843 and 873 of SEQ ID NO.:1.

Also in accordance with the present invention, the first and second annealing arm may each independently have from about 9 to 15 deoxyribonucleotides.

Again in accordance with the present invention, the first and second annealing arms may be totally (100%) complementary to the HCV region or may possess one, two or three nucleotides which are not complementary to the HCV region.

In accordance with a specific embodiment of the present invention, when the deoxyribozyme possesses one nucleotide which is not complementary to the HCV region it preferably does not consist in SEQ ID NO.:66. However, other aspects of the invention may include SEQ ID NO.:6.

In accordance with the present invention, the deoxyribozyme may be capable of intracellular cleavage of a HCV sequence.

The HCV sequence may be, for example, a HCV genome or a portion thereof.

Also in accordance with the present invention, the deoxyribozyme may be capable of cleaving a HCV sequence found in a mammal (an HCV-infected mammal).

Further in accordance with the present invention, the HCV sequence may be a HCV genome or a portion thereof (e.g. a replication intermediate).

In accordance with the present invention, the deoxyribozyme may be, for example, of from about 25 to about 55 deoxyribonucleotides long or from about 30 to about 50 deoxyribonucleotides long or from about 30 to about 40 deoxyribonucleotides long or less.

In accordance with an exemplary embodiment of the present invention the DNAzyme may comprise SEQ ID NO.:71 or SEQ ID NO.:73. In accordance with a further exemplary embodiment of the present invention the DNAzyme may consist in SEQ ID NO.:71 or SEQ ID NO.:73.

Also in accordance with a particular embodiment of the present invention, a sequence comprising or consisting of SEQ ID NO.:71 or SEQ ID NO.:73 may have (in the first and/or second annealing arm) at least one nucleotide which is not complementary to SEQ ID NO.:1. More particularly, a sequence comprising or consisting of SEQ ID NO.:71 or SEQ ID NO.:73 may have one nucleotide which is not complementary to SEQ ID NO.:1 in either one of the first or second annealing arm).

Further in accordance with the present invention, a sequence comprising or consisting of SEQ ID NO.:71 or SEQ ID NO.:73 may have other nucleotides or base which are modified.

In accordance with the present invention, the deoxyribozyme may comprise at least one phosphorothioate-derivative nucleotide.

Further in accordance with the present invention, the deoxyribozyme may comprise at least one 2′-O-methyl nucleotide analog.

Also in accordance with the present invention, the deoxyribozyme may comprise at least one morpholino-derivative nucleotide.

It is to be understood herein that one or more (unmodified) nucleotides may be replaced by a modified nucleotide (also referred herein as a nucleotide derivative or analog) as described herein without substantially affecting the activity of the deoxyribozyme of the present invention. The modified nucleotide may be inserted in place of an original nucleotide (unmodified nucleotide) in one or both of the annealing arm or sometimes within the catalytic region.

The nucleotide derivative or nucleotide analog may be located, for example, at one or both ends of the deoxyribozyme. In addition, the nucleotide derivative or nucleotide analog may be located within the first and/or second arm of the deoxyribozyme.

In accordance with the present invention, the target HCV core-encoding region may be, for example, a messenger RNA or a genomic RNA.

Also in accordance with the present invention, the target HCV core-encoding region may be, for example, single-stranded.

The catalytic region of the deoxyribozyme may comprise, for example, a type I domain (SEQ ID NO.: 2) or a type II (SEQ ID NO.: 3) domain or any catalytic domain (variant) able to cleave a nucleotide sequence found within a target sequence.

In accordance with the present invention, the first or second annealing arm may comprise, for example, at least one nucleotide which is not substantially complementary to the target HCV core-encoding region. For example, the first or second annealing arm may comprise one or two nucleotides which are not substantially complementary to the target HCV core-encoding region.

Additionally, upon hybridization of the deoxyribozyme and the target to form a complex, the complex may comprise an unpaired purine (the purine may be located in the target) followed by a paired pyrimidine located at the junction between the first and second annealing arms as illustrated herein.

In a further aspect, the present invention provides a deoxyribozyme which may be able to cleave a target HCV core-encoding region. The deoxyribozyme may comprise the formula: X₁-Cₐ-X₂, where X₁ may be a first annealing arm having, for example, a nucleotide sequence of from 7 to 20 deoxyribonucleotides, Cₐ may be a type I or type II catalytic domain or a variant thereof and X₂ may be a second annealing arm having, for example, a nucleotide sequence of from 7 to 20 deoxyribonucleotides.

More particularly, the present invention provides a deoxyribozyme which may be capable of intracellularly cleaving a target HCV core region, the deoxyribozyme may comprise the formula X₁-Cₐ-X₂, wherein X₁, Cₐ and X₂ are as defined above and wherein the deoxyribozyme may be substantially complementary to a HCV sequence which may be located between nucleotides 330 and 370 of SEQ ID NO.:1, or between nucleotides 676 and 715 of SEQ ID NO.:1, or between nucleotide 835 and 880 of SEQ ID NO.:1.

In accordance with the present invention, the first annealing arm may, more particularly comprise, for example from 9 to 15 deoxyribonucleotides (inclusively).

Also in accordance with the present invention, the second annealing arm may, more particularly comprise, for example, from 9 to 15 deoxyribonucleotides (inclusively).

Further in accordance with the present invention, the deoxyribozyme may be selected, for example, from the group consisting of:
a deoxyribozyme which may comprise a nucleotide sequence defined herein (e.g., FIG. 1A to 1E or defined in the sequence listing),
a deoxyribozyme which may consist of a nucleotide sequence defined herein (e.g., FIG. 1A to 1E or defined in the sequence listing), and;
a deoxyribozyme analog of any one nucleotide sequence defined herein (e.g., FIG. 1A to 1E or defined in the sequence listing).

In accordance with the present invention, the deoxyribozyme analog may have, in the first and/or second annealing arm, one or two nucleotides (modified (nucleotide analog) or not (A, T, G, C)) which are not complementary to the target HCV core-encoding region.

In accordance with the present invention, the nucleotides found in the deoxyribozyme of the present invention may be deoxyribonucleotides.

In an additional aspect, the present invention provides a composition, such as, for example, a pharmaceutical composition, which may comprise:
at least one deoxyribozyme as defined herein and combination thereof, and
a (pharmaceutically acceptable) carrier.

In accordance with the present invention, the composition may be used, for example, for the treatment of a HCV infected individual (reduction of viral load). More particularly, the present invention relates to method of treatment of HCV related disease, such as for example, hepatitis (acute or chronic), HCV-related cirrhosis, HCV-related cancer (e.g., hepatocellular carcinoma) etc.

Also in accordance with the present invention, the deoxyribozyme may be used, for example, for the treatment of an individual (mammal) having or susceptible of having a HCV infection.

In a further aspect, the present invention relates to the use of a deoxyribozyme described herein and combination thereof, in the manufacture of a medicament (drug, composition, pharmaceutical composition) for the treatment of a HCV infection.

The present invention also relates to the use of the deoxyribozyme described herein in the manufacture of a medicament for the prevention (e.g., partial prevention) or treatment of HCV infection or a HCV-related disease.

In yet a further aspect, the present invention relates to a method of treating an individual (mammal) having or susceptible of having a HCV infection and/or an HCV-related disease. In accordance with the present invention, the method may comprise administering a deoxyribozyme as described herein or combination thereof or a composition as described herein to the individual (mammal).

In an additional aspect, the present invention relates to the use of a HCV core region substantially conserved among HCV subtypes in the generation of deoxyribozymes.

In accordance with the present invention, the target HCV core-encoding region may comprise or consist of the HCV sequence identified herein (e.g., FIG. 1A or defined in the sequence listing).

More particularly, the present invention relates to the use of a HCV sequence located between nucleotides 330 and 370 of SEQ ID NO.:1, or between nucleotides 676 and 715 of SEQ ID NO.:1, or between nucleotide 835 and 880 of SEQ ID NO.:1 in the generation of a deoxyribozyme which may be able to bind (and which may also be able to cleave (e.g., intracellularly) a HCV genomic sequence, a HCV replication intermediate or portion thereof.

In accordance with an exemplary embodiment of the invention, the HCV sequence may be selected, for example, from the group consisting of SEQ ID NO.:16, SEQ ID NO.:19, SEQ ID NO.:22 and SEQ ID NO.:25 or a portion thereof.

In accordance with an embodiment of the present invention, the portion of SEQ ID NO.:16, SEQ ID NO.:19, SEQ ID NO.:22 or SEQ ID NO.:25 may be one which comprises, for example, a sequence of about 15 nucleotides which may have a A/U or A/C predicted cleavage site therein (e.g., at about 7 to 8 nucleotides (or more) from either the 5′-end or 3′-end).

Also in accordance with the present invention, the HCV sequence may consists in SEQ ID NO.:16, SEQ ID NO.:19, SEQ ID NO.:22 or SEQ ID NO.:25 or may consists in a portion of SEQ ID NO.:16, SEQ ID NO.:19, SEQ ID NO.:22 or SEQ ID NO.:25 which may have, for example, a sequence of about 15 nucleotides long with a A/U or A/C predicted cleavage site therein (e.g., at about 7 to 8 nucleotides (or more) from either the 5′-end or 3′-end).

In accordance with the present invention, the deoxyribozyme may be generated by using a target with one nucleotide which is not complementary to the HCV sequence (SEQ ID NO.:1). In accordance with a specific embodiment of the invention the HCV sequence preferably does not consists in: CUUUCUCUAUCUUCCUC (SEQ ID NO.:54).

In an additional aspect, the present invention relates to a method of generating a deoxyribozyme, which may comprise a step of allowing synthesis of or synthesizing (using chemical synthesis or biological synthesis (e.g., recombinant technology)) a deoxyribozyme which may comprise formula X₁-Cₐ-X₂, where X₁ may be a first annealing arm which may have a nucleotide sequence of from 7 to 20 deoxyribonucleotides, Cₐ may be a type I or type II catalytic domain and X₂ may be a second annealing arm which may have a nucleotide sequence of from 7 to 20 deoxyribonucleotides. In accordance with an embodiment of the present invention, the deoxyribozyme may be substantially complementary to a HCV sequence located, for example, between nucleotides 330 and 370 of SEQ ID NO.:1, or between nucleotides 676 and 715 of SEQ ID NO.:1, or between nucleotide 835 and 880 of SEQ ID NO.:1.

In accordance with the present invention, when the synthesis is done chemically the deoxyribonucleotide may comprise at least one modified nucleotide or at least one deoxyribonucleotide may be replaced with a nucleotide analog.

A biological synthesis method may entail, for example, providing a vector (or a suitable portion having a promoter) encoding the desired deoxyribozyme sequence for performing cell-free assay or transforming a cell with a suitable vector for performing intracellular synthesis of the desired deoxyribozyme.

Pharmaceutically acceptable acid (addition) salts of the deoxyribozymes may be prepared by methods known and used in the art and are encompassed by the present invention.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carriers” or “pharmaceutical carriers” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's orfixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

A “fragment” is to be understood herein as an oligonucleotide originating from a portion of an original or parent sequence. Fragments encompass oligonucleotides having truncations of one or more nucleotides, wherein the truncation may originate from the 5′-end or the 3′-end. Biologically active fragments are encompassed by the present invention.

A “deoxyribozyme analog” may have sequence similarity with that of an original sequence or a portion of an original sequence and may also have a modification of its structure as discussed herein. A “deoxyribozyme analog” is to be understood herein as a molecule having a biological activity and chemical structure similar to that of a deoxyribozyme described herein. An analog comprises a deoxyribozyme which may have, at least 70%, 80%, 90% or 95% sequence identity with an original sequence or a portion of an original sequence. Also, an “analog” may have, for example, at least 70%, 80%, 90% or 95% sequence identity to an original sequence and may include nucleotide analogs.

TABLE 1
Abbreviations

| | |
|---|---|
| Dz | Deoxyribozyme |
| mtDz | mutant deoxyribozyme |
| Nt | Nucleotide |
| RT-PCR | Reverse transcriptase polymerase chain reaction |
| UTR | Untranslated region |

It is to be understood herein, that if a “range” or “group of substances” is mentioned with respect to a particular characteristic (e.g., temperature, concentration, time and the like) of the present invention, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-range or sub-group encompassed therein; and similarly with respect to any sub-range or sub-group therein. Thus, for example,
with respect to a length of 40 nucleotides (bases) long or less, is to be understood as specifically incorporating herein each and every individual length, e.g., a length of 15, 20, 25, 32, 39, etc.; therefore, unless specifically mentioned, every range mentioned herein is to be understood as being inclusive. For example, the expression from 15 to 40 nucleotides long, is to be understood as including 15 and 40;
with respect to the term “a region located between nucleotide 835 and nucleotide 880” and similar terms, is meant to include each possible and individual ranges for example, embodiments of ranges encompassed herewith may include; 836 to 880, 836 to 880, 837 to 880, 835 to 879, 835 to 878, 835 to 877, 835 to 876, 836 to 876, 840 to 870, 845 to 875, 844 to 874, 843 to 873, and so on. An exemplary limitation of a range may be, for example, that it may not preferably define a range lower than 14 nucleotides long;
and similarly with respect to other parameters such as sequences, other length, concentrations, elements, etc. . . .

It is in particular to be understood herein that the sequences, regions, portions defined herein each include each and every individual sequences, regions, portions described thereby as well as each and every possible sub-sequence, sub-region, sub-portion whether such sub-sequences, sub-regions, sub-portions are defined as positively including particular possibilities, as excluding particular possibilities or a combination thereof; for example an exclusionary definition for a region may read as follows: “provided that said sequence is no shorter than 10, 11, 12, 13, 15, 20 nucleotides. Yet a further example of a negative limitation is the following; a sequence comprising SEQ ID NO.: X with the exclusion of the sequence defined in SEQ ID NO. Y; etc. An additional example of a negative limitation is the following; provided that said sequence is not SEQ ID NO.:Z. Yet further negative limitations encompassed herewith may include, for example, “provided that said DNAzyme does not comprise a 3′-terminal inverted deoxyabasic moiety” or “provided that said DNAzyme does not comprise an abasic moiety of formula defined in U.S. patent application Ser. No. 09/817,879 to Blatt et al.

Another exemplary embodiment of a negative limitation with respect to deoxyribozymes may be the following “provided that said deoxyribozyme does not consist in “GACGAAGA GGCTAGCTACAACGAAGAGAAAG” (SEQ ID NO.:66) or in “GTTTAGGA GGCTAGCTACMCGATCGTGCTC” (SEQ ID NO.:67) or in “TCACCTTA GGCTAGCTACAACGA CCAAGTTA” (SEQ ID NO.:68). However the above mentioned deoxyribozymes may or may not be excluded from some of the pharmaceutical compositions, uses and/or methods described herein.

### BRIEF DESCRIPTION OF THE DRAWINGS

In drawings which illustrate exemplary embodiments of the invention,

FIGS. 1A to 1E represent lists of target HCV core region and DNAzyme targeting such regions;

FIG. 2 is a schematic map of the HCV RNA genome and deoxyribozyme recognition sites. Coding regions for structural (open rectangles) and non-structural (NS) (grey rectangles) viral proteins along with protein cleavage sites by cellular signal peptidases (open diamonds), virally encoded proteases (closed diamonds) and predicted deoxyribozyme recognition sites within the core (C) open reading frame (arrow) are shown (Top). Nucleotide sequence 1 to 976 from HCV type 1b [21] encoding the 5′UTR and the virus core protein (bottom) is also provided. The AUG initiation codon for the HCV polyprotein is shown in bold. Deoxyribozyme recognition sites are underlined. The six-nucleotide extension for the 5′ arm of Dz858-15-15 is shown by the dashed underline. The predicted deoxyribozyme cleavage site is indicated for each deoxyribozyme by the solid triangles,

FIG. 3 represents the in vitro cleavage of HCV RNA spanning HCV UTR-core genomic position 1 to 976 by Dz348-9-15end, Dz699-9-15end, Dz858-15-15end, Dz858-9-15end and mtDz858-9-15end. Reactions were performed at 37° C. for 1 h as detailed in Materials and Methods. Deoxyribozyme to HCV RNA (100 nM) ratios ranged from 0.1 to 1000. Full-length HCV RNA (HCV RNA) or HCV RNA cleavage products produced after Dz348-9-15end (628 and 348 nucleotides), Dz699-9-15end (699 and 277 nt), Dz858-15-15end (858 and 118 nt) treatment are indicated. Uncut HCV RNA following mtDz858-9-15end treatment (S:E ratio of 1:1000) is noted, gel top right,

FIG. 4 illustrates the comparison of catalytic activity of the four deoxyribozymes. Effect of different concentrations of deoxyribozymes on in vitro cleavage of HCV RNA (a). HCV UTR-core RNA was incubated with increasing molar concentrations of deoxyribozymes. Resulting cleavage products were resolved by gel electrophoresis and quantified by phosphorimaging. Results from three independent experiments were plotted as the percent HCV RNA cleavage±SEM versus deoxyribozyme concentration ratios. Time course of Dz858-9-15end and Dz858-15-15end cleavage of HCV RNA (b). HCV UTR-core RNA (1 μg) was incubated with deoxyribozyme (S:E 1:10) at 37° C. for up to 90 minutes. Resulting cleavage products were resolved by gel electrophoresis and quantified by phosphorimaging. The results from three separate experiments were plotted as the percent HCV RNA cleavage±SEM versus time,

FIG. 5 represents the intracellular cleavage of HCV core protein RNA by Dz858-15-15end. 293rtTA and HuH-7 cells were transfected with pHCV-UTR-core plasmid in the presence of a 1000-fold molar excess of Dz858-15-15end or mtDz858-15-15end. After 24 hours, total RNA was extracted and processed for quantitative RT-PCR. A summary graph of the percent HCV RNA remaining after deoxyribozyme treatment for 293rtTA (left) or HuH-7 (right) cells treated with Dz858-15-15end (solid bar) or mtDz858-15-15end (open bar) is shown.*, p≦0.05 by Mann-Whitney Rank Sum test, n=3 independent transfection experiments,

FIG. 6 is a schematic illustrating the structure of exemplary embodiments of oligomers with phosphate (DNA), phosphorothioate-, peptide nucleic acid (PNA), morpholino-, 2′-O-methoxyethyl (MOE) and 2′-O-methyl (2OMe) backbones,

FIG. 7 is a schematic illustrating the general structure of type I and type II deoxyribozymes R=A or G and Y=U or C;

FIG. 8 represents in vitro cleavage of HCV using O-methyl variants at concentrations higher than 23 nM;

FIG. 9A is a schematic of Dz858 cleavage target and FIG. 9B represents in vitro cleavage of HCV RNA by Dz858 variants at a concentration of 23 nM. The plot of % RNA cleavage+SEM versus deoxyribozyme concentration was based on three independent experiments.

FIG. 10 Expression of HCV proteins in Huh-7 cells following transfection of synthetic, genomic-length HCV RNA. Picture of a 0.8% agarose-formaldehyde gel of the full-length genomic HCV RNA, stained with ethidium bromide (A). Huh-7 cells either sham transfected (Mock), transfected with HCV RNA (HCV RNA) or with HCV core protein eukaryotic expression plasmid (pHCV-core). HCV protein expression was measured after 24 h of cell culture either by immunofluorescence (B) or after equal amounts of Huh-7 cellular protein was processed for SDS-PAGE and immunoblot (C).

FIG. 11 Reduction in intracellular HCV RNA (A), HCV core protein (B) and HCV antigen reactivity (C) in Huh-7 cells following treatment with Dz858-4-OMe. HCV RNA+SEM was plotted from four independent experiments following total RNA normalization with β-actin RNA. Huh-7 cells were harvested 24 h (pre- and co-addition) or 36 h (post-addition) following treatment and processed for anti-HCV or anti-actin immunoblot (B) or HCV immunostain and quantitative digital imaging (C).

FIG. 12 is a schematic illustrating exemplary structure of a Dz858 morpholino-variant, and;

FIG. 13 is a histogram expressing the percent HCV RNA signal. Tissue samples taken at 18 hours from 9 mice following mock injection or injection with 293 cells containing genomic HCV RNA or with 293 cells containing genomic HCV RNA+phosphorothioate Dz858-15-15 were assayed for the level of HCV RNA by RT-qPCR. Tissue sample HCV RNA was normalized using the housekeeping gene GAPDH. HCV RNA signal levels seen following RT-qPCR analysis were given an arbitrary value of 100 percent.

### DETAILED DESCRIPTION OF THE INVENTION

### Materials and Methods

### Deoxyribozyme Design and Construction

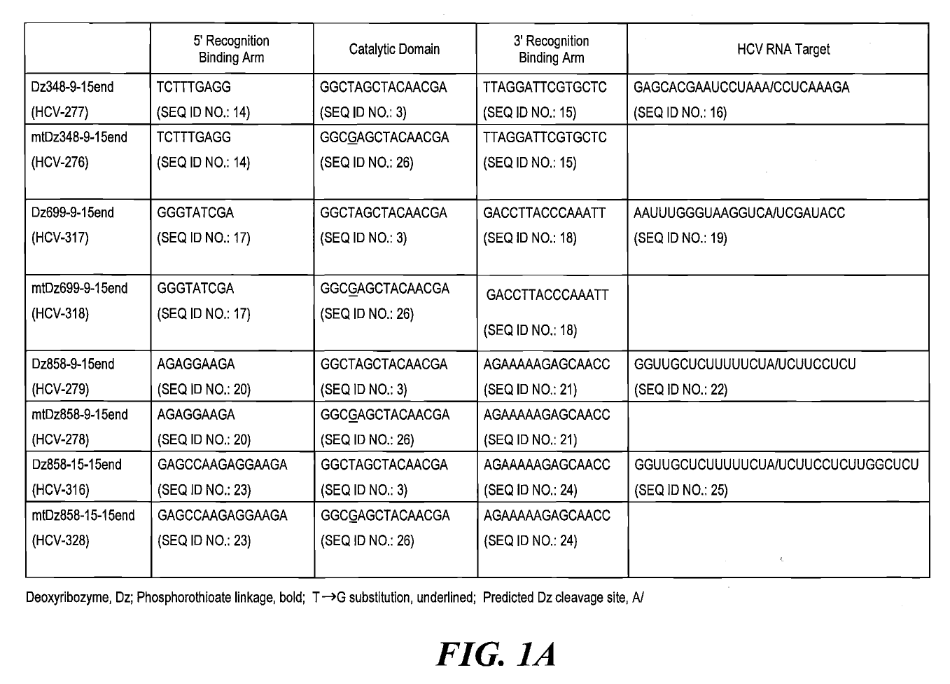

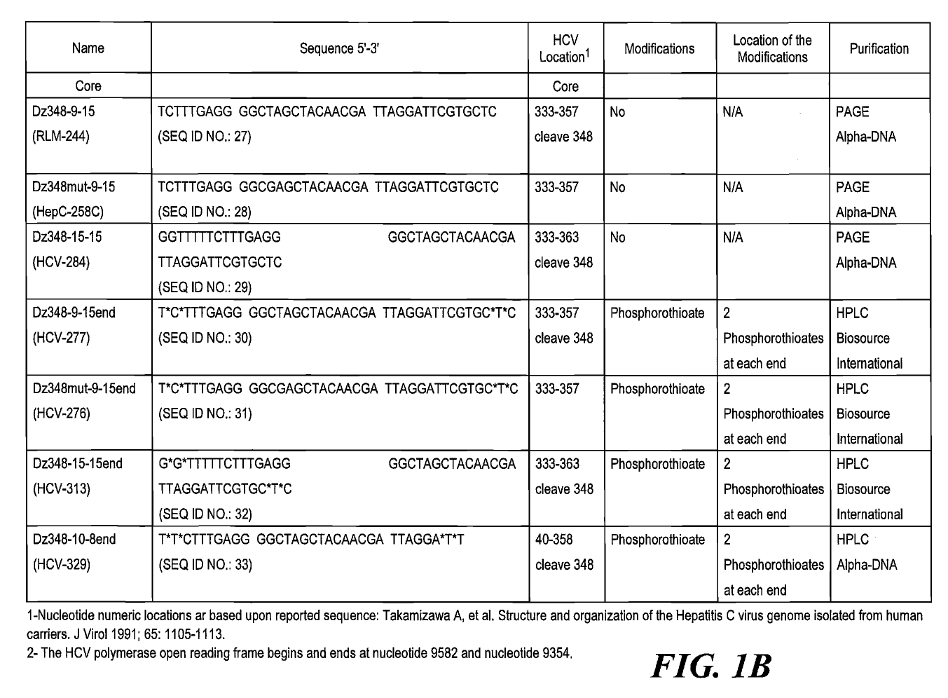

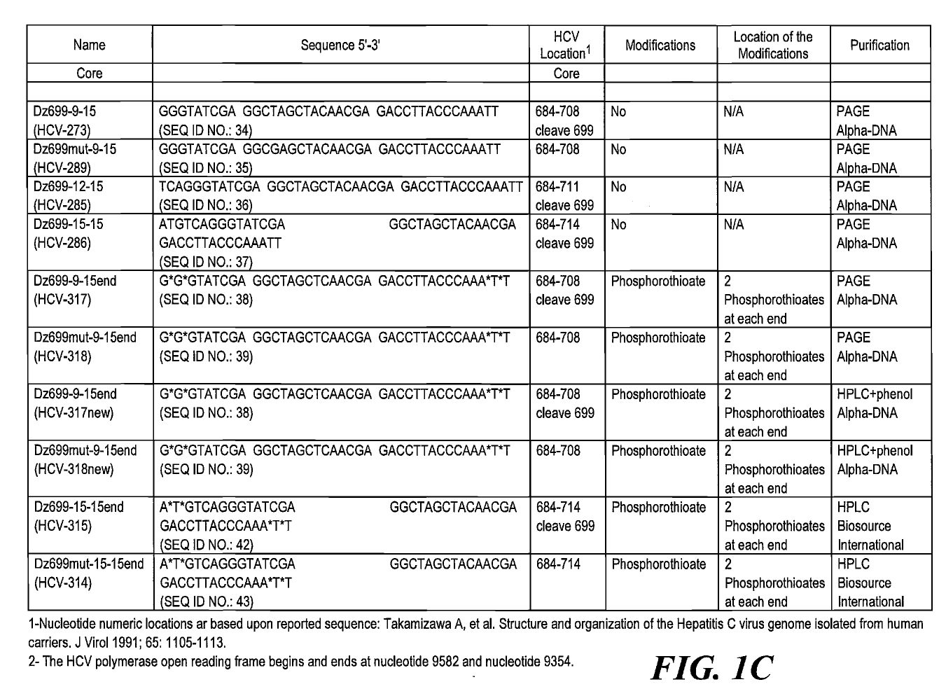

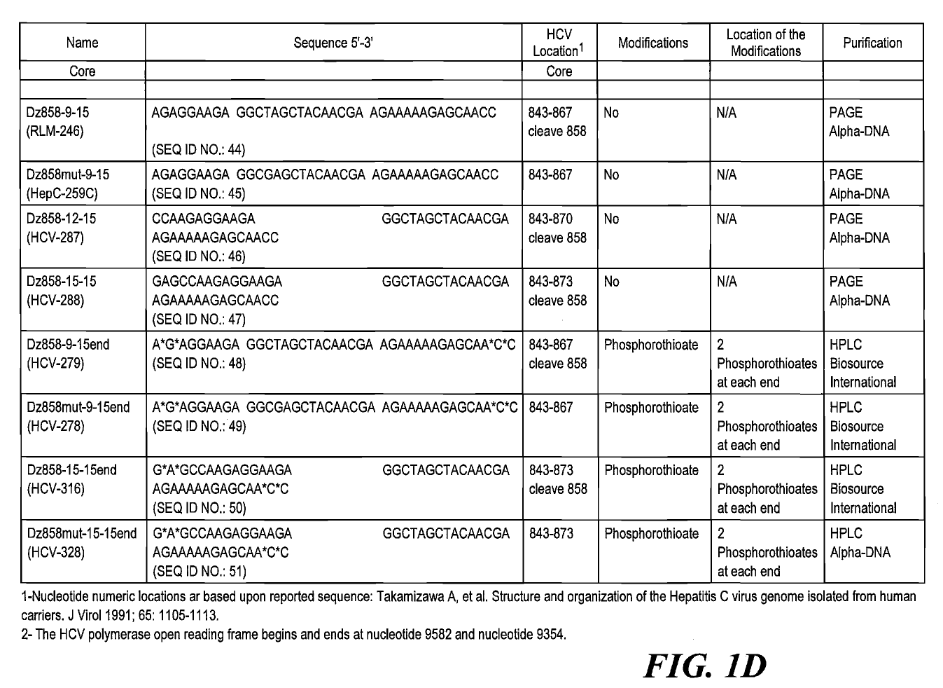

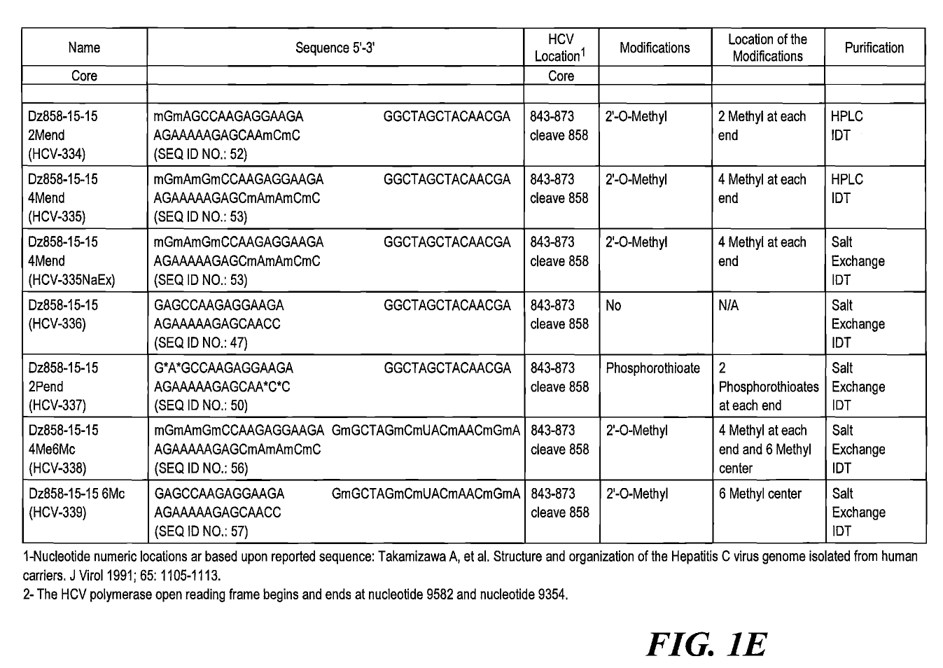

The HCV type 1 genomic segment encompassing the contiguous 5′-untranslated region (UTR) and core protein coding sequence (contained within pGEM-7Zf-HCV) was used in this experiment. The cDNA sequence of HCV may be found for example in Takamizawa A, et al., 1991 [21] and in Genbank under accession number M58335. The sequence was surveyed using the m-fold computer program (www.bioinfo.rpi.edu/applications/mfold) to identify single-stranded loops within this HCV segment having deoxyribozyme cleavage potential [22,23]. Type II deoxyribozymes used in the present study and listed in FIG. 1A to 1E were synthesized using phosphoramidite chemistry (Alpha DNA Ltd., Montreal, QC and Biosource International, Camarillo, Calif.) and high pressure liquid chromatography. Type I deoxyribozymes may be synthesized in a similar manner. To compare the effect of deoxyribozyme arm length on cleavage efficiency, deoxyribozymes of varying arm lengths were also synthesized. The 3′ recognition arm was fixed to a length of 15 nucleotides and the 5′ recognition arm was varied to either 9 or 15 nucleotides [24]. Mutant deoxyribozymes (mtDz) unable to cleave HCV RNA targets were generated by substituting a guanine for a thymidine residue at position 4 of the catalytic domain [7].

### Synthesis of the Deoxyribozyme RNA Substrate

To generate sufficient HCV RNA target substrate the cDNA sequence from pGEM-7Zf-HCV spanning the HCV 5′UTR and the adjoining core protein coding sequence (HCV genome positions 1 to 976) (Takamizawa A, et al., 1991 [21]) was amplified by polymerase chain reaction (PCR) using the sense primer 5′-TGTAATACGACTCACTATAGCGA-3′ (SEQ ID NO.:4) encoding the bacteriophage T7 RNA polymerase promoter and an anti-sense HCV-encoding primer 5′-TCATACACAATGCTTGCGTTG-3′ (SEQ ID NO.:5). Amplified DNA was fractionated by agarose gel electrophoresis and purified using the QIAquick gel extraction kit (Qiagen Inc., Mississauga, ON). Radiolabeled HCV RNA substrate was generated as recommended by the manufacturer using 1 μg amplified HCV cDNA, the MegaScript T7 transcription kit (Ambion Inc., Austin, Tex.), T7 RNA polymerase (Ambion Inc.) and [³²P]UTP (20 mCi/ml, 800 Ci/mmole) (Amersham, Piscataway, N.J.). Transcription reactions were performed at 37° C. for 6 h followed by DNA template removal using RNAse-free DNAse (Ambion Inc.), phenol-chloroform extraction and ethanol precipitation.

### Kₘ and K_{cat} and Time Course Determinations

The Kₘ and K_{cat} values for deoxyribozymes were determined using the Michaelis-Menten enzyme equation Y=(Vₘₐₓ*X)/(Kₘ+X) and the equation K_{cat}=Vₘₐₓ/Sₜ, respectively, where the Vₘₐₓ was obtained empirically, Y represents the % cleavage, X represents the deoxyribozyme concentration and Sₜ represents the original substrate concentration of 100 nM (Prism 3.03 software, GraphPad Software Inc., San Diego, Calif.). A total amount of 100 nM radiolabeled HCV RNA substrate was suspended in 50 mM Tris-HCl buffer pH 7.5 containing 10 mM MgCl₂ and incubated for 1 h at 37° C. with increasing log₁₀ concentrations of deoxyribozyme ranging in value from 10 nM to 100 μM. Cleavage reactions were terminated by the addition of gel loading buffer containing 95% formamide, and RNA cleavage products resolved by gel electrophoresis in a 6% polyacrylamide gel containing 8 M urea and Tris-borate buffer [25]. Following electrophoresis, gels were dried and cleavage products quantified using the Si Phosphorlmager (Molecular Dynamics, Sunnyvale, Calif.). Relative band intensity for the cleavage products was plotted as the percentage of cleaved RNA versus the deoxyribozyme concentration.

An amount of 100 nM radiolabeled HCV RNA substrate was also incubated at 37° C. with 1 μM deoxyribozyme in 50 mM Tris-HCl buffer pH 7.5 and 10 mM MgCl₂ for 0 to 90 min. Reactions were stopped by the addition of gel loading buffer and cleavage products resolved by gel electrophoresis for band quantification as described above. The percentage of cleavage product versus time was then plotted.

### Analysis of the Intracellular Cleavage of HCV RNA

The HCV eukaryotic expression plasmid, pHCV-UTR-core, encoding a 942 base RNA segment of the HCV UTR and core protein coding sequence (HCV genome position 38 to 980) was constructed by PCR amplification of pGEM-7Zf-HCV plasmid using the sense primer 5′-cccaagcttggGTGAGGMCTACTGTCTTC-3′ (SEQ ID NO.:6) and the antisense primer 5′-ttaagcggccgcaaatcTGCCTCATACACA-3′ (SEQ ID NO.:7). Sequences “cccaagcttgg” (SEQ ID NO.:40) and “ttaagcggccgcaaatc” (SEQ ID NO.:41) found within the above-mentioned primers, comprise a HindIII and Not I restriction sites, respectively (shown in italics). These restriction sites and flanking sequences were generated to attain a recommended annealing temperature of 55° C. during PCR amplification and to clone the resulting PCR product in proper orientation into the eukaryotic expression vector pcDNA3.1 (+). These primer sequences contain sufficient G:C to A:T ratios of ˜50% for proper annealing at 55° C. during PCR synthesis and one “TAA” stop codon upstream of the Not 1 site for proper termination of synthetic HCV RNA during cellular synthesis. Amplified DNA was purified by agarose gel fractionation and QIAquick gel extraction kit (Qiagen Inc.), followed by Hind III and Not I restriction enzyme digestion and insertion into the multi-cloning site of the eukaryotic expression vector, pcDNA3.1 (+) (Invitrogen Inc., Burlington, ON).

The human hepatoma cell line HuH-7, kindly provided by Dr Tatsuo Takahashi (Health Science Research Resources Bank, Japan. Nakabayashi H, Taketa K, Miyano K, Yamane T, Sato J. Growth of human hepatoma cells lines with differentiated functions in chemically defined medium. Cancer Res. 1982 Sept; 42(9):3858-63; available from the Japanese Collection of Research Bioresources cell line distribution center (Tokyo, Japan); Cat. No. JCRB0403) was cultured in Dulbecco's Modified Eagles Medium (DMEM) (Invitrogen) supplemented with 10% fetal bovine serum (FBS) (Medicorp Inc., Montreal, Qc). The human embryonic kidney cell line 293rtTA was kindly provided by Dr Bernard Massie (Biotechnology Research Institute, Montreal, Qc, Massie B, Couture F, Lamoureux L, Mosser D D, Guilbault C, Jolicoeur P, Belanger F, Langelier Y. Inducible overexpression of a toxic protein by an adenovirus vector with a tetracycline-regulatable expression cassette. J. Virol. 1998 March; 72(3):2289-96. and from American Type Culture Collection, Manassas, Va., CRL-1573), and cultured in DMEM medium supplemented with 10% tetracycline-free FBS (Clonetech, Palo Alto, Calif.). Both cell lines typically exhibited transfection efficiencies of 50 to 60% when tested with the green fluorescent protein expression plasmid, pCMV:GreenLantern (Invitrogen Inc, and JT data not shown).

293rtTA cells seeded at 8.5×10⁵ cells per well or HuH-7 cells seeded at 4×10⁵ cells per well in 6-well plates were cultured overnight and co-transfected with pHCV-UTR-core and deoxyribozyme at a DNA to deoxyribozyme molar ratio of 1:1000 using 3 μg of Lipofectamine 2000 per 1 μg of total DNA (Invitrogen Inc.). In order to equalize HCV plasmid DNA concentration (2.5 μg final DNA concentration) between transfection experiments, final plasmid DNA concentrations were adjusted to 2.5 μg using vector DNA. After 6h, cells were placed in complete medium and cultured for an additional 18h. Total cellular RNA was then extracted with Oligotex direct mRNA extraction kit (Qiagen Inc.) and treated with DNAse for 3 h to remove transfected plasmid DNA (Ambion Inc.).

The degree of HCV RNA cleavage was determined by quantitative RT-PCR using HCV-derived sense primer 5′-AAGGCCTTGTGGTACTGCCTGATA-3 (SEQ ID NO.:8)′, 6′-carboxyfluorescein, succinimidyl ester (FAM)-labeled probe, having sequence: 5′-FAM/ACCGTGCACCATGAGCACGMTCCTAA/3′Iowa Black FQ-3′ (SEQ ID NO.:9) and antisense primer 5′-GGCGGTTGGTGTTACGTTTGGTTT-3′ (SEQ ID NO.:10). The DNA signal generated from HCV RNA target was normalized to the neomycin resistance cDNA gene generated from the open reading frame found within our eukaryotic expression plasmid, pHCV-UTR-core. The neomycin resistance gene cDNA was quantified using sense primer 5′-ACCTTGCTCCTGCCGAGAAAGTAT-3′ (SEQ ID NO.:11), 5′ cyanine-5 (5Cy5)-labeled probe having sequence: 5′-5Cy5/AATGCGGCGGCTGCATACGCTTGAT/-3′IowaBlack FQ (SEQ ID NO.:12) and antisense primer 5′-CGATGTTTCGCTTGGTGGTCGAAT-3′ (SEQ ID NO.:13). Primers and probes were designed and synthesized by Integrated DNA Technologies Inc. (Coralville, Iowa). Primers and probes were used at final concentrations of 400 nM and 200 nM for the amplification of HCV and neomycin resistance gene cDNAs, respectively. cDNAs were initially suspended in Brilliant Multiplex QPCR master mix containing carboxy-x-rhodamine succinimidyl ester (ROX) reference dye (Stratagene Inc., La Jolla, Calif.), followed by heating for 10 minutes at 95° C., and 45 amplification cycles. Each amplification cycle consisted of a 15 sec incubation at 95° C. and a 1 min annealing and elongation step at 60° C. cDNAs were amplified and quantified using the Mx3000P real-time PCR thermocycler (Stratagene Inc.). Logarithmic concentrations of pHCV-UTR-core plasmid ranging from 1 pg to 10 ng served as reference standards for both the HCV and neomycin resistance gene cDNAs.

### Computer Analysis

Selection of deoxyribozyme annealing arms was performed using Vector NTI 8.0 software (Informax, Fredrick, Md.) [26,27]. Mann-Whitney Rank Sum Test was performed using Sigma Stat 3.0 statistical software package for Windows (Aspire Software International, Leesburg, Va.).

### EXAMPLES

### Example 1

### Design of Phosphorothioate-Based Deoxyribozymes

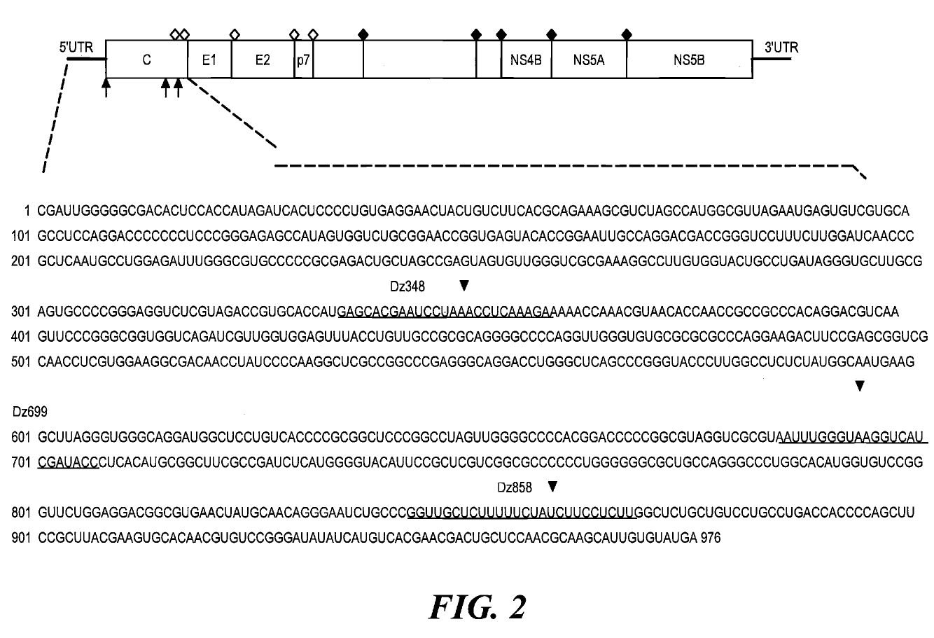

Initially, we designed deoxyribozymes that targeted highly conserved RNA sequences contained within the HCV core protein coding region to lessen the likelihood that our deoxyribozyme candidates would eventually be found ineffective against de novo HCVs undergoing continuous mutagenesis [30]. Using the m-fold program (M. Zuker. Mfold web server for nucleic acid folding and hybridization prediction. Nucleic Acids Res. 31 (13): 3406-15, (2003) & D. H. Mathews, J. Sabina, M. Zuker & D. H. Turner. Expanded sequence dependence of thermodynamic parameters improves prediction of RNA secondary structure. J. Mol. Biol. 288: 911-940 (1999)) to predict HCV core RNA secondary structures and potential sites for deoxyribozyme hybridization and cleavage [31,32], we designed and synthesized three sets of Type II deoxyribozymes having asymmetric arms and phosphorothioate linkages incorporated at the flanking end of the two recognition arms (see FIG. 1A to 1E and FIG. 2). Arm asymmetry and incorporation of phosphorothioate linkages were investigated in order to determine whether this would enhance deoxyribozyme catalysis and increase their half-life [33, 34]. Deoxyribozymes were designed to recognize HCV RNA at positions 335 to 359 (target HCV: SEQ ID NO.:16) (Dz348-9-15 (DNAzyme: SEQ ID NO.: 27)), 684 to 708 (target HCV: SEQ ID NO.: 19) (Dz699-9-15 (DNAzyme: SEQ ID NO.:34)) and 843 to 873 (target HCV: SEQ ID NOs.: 22 and 25) (e.g., Dz858-9-15 (DNAzyme: SEQ ID NO.:44)). We also synthesized identical deoxyribozyme sets but with ablated catalytic sites to serve as negative controls (i.e., mtDz348-9-15 (SEQ ID NO.:28), mtDz699-9-15 (SEQ ID NO.:34) and mtDz858-9-15 (SEQ ID NO.:45)). Mutated deoxyribozyme (e.g., mtDz858-9-15) constructs were expected to allow us to distinguish non-cleavage-specific activity when comparing overall decreases in HCV RNA signal [24].

Previous studies which examined the secondary structure of HCV RNA indicate that RNA folding may influence the accessibility of antisense oligonucleotides to their HCV RNA counterparts [30, 38]. This issue may therefore be further compounded by the major and subtle structural differences seen among the various HCV subtypes and quasispecies [37]. We have therefore attempted using the above-mentioned technology to avoid this possible pitfall by designing deoxyribozymes which recognize highly conserved regions contained within the HCV core protein coding sequence, as well as limiting our choice of deoxyribozymes to only those candidates which recognize conserved open structures found among the large repertoire of reported HCV genome sequences. We observed that the annealing arms of Dz858-15-15 (SEQ ID NO.:47) annealing to target HCV SEQ ID NO.:25 appeared identical (100% homologous) to 36 of the 100 HCV sequences listed in the National Center for Biotechnology Information (NCBI), USA databank (available at www.ncbi.nlm.nih.gov on Sep. 10, 2004), or differ by only one base pair for the remaining 64 listed sequences. The 100 RNA sequences that were homologous to Dz858-15-15 and contained within the NCBI databank listed sequences for three HCV subtypes (i.e. 1b, 2, and 4) as well as numerous viral strain variants. Thus our current Dz858-15-15 construct or ones which bear a single alternative nucleotide sequence should be able to recognize a broad range of HCV sequences, with a lessened possibility of inactivity due to limited recognition of HCV subtypes or the presence of a single mutational variation within the HCV RNA target.

Similar database comparison of some of our DNAzymes constructs were made on or around Jul. 28, 2006 using HCV DATABASES (http://hcv.lanl.gov/content/hcv-db/index) web based BLAST search using a sequence published in U.S. application Ser. No. 09/817,879 to Blatt et al. or a corresponding portion of Dz855-9-15. BLAST was made using a 100% sequence match as the selection criteria.

The BLAST results indicate that, overall, Dz858 recognized 26% of all HCV genotypes recovered from the HCV database versus 59% for the published sequence. However, results for individual genotypes varied. For example, Dz858 matched over 30% of the HCV genotypes for types 1,1b, versus over 60% matched for the published sequence. Dz858 matched 50% of HCV type 2 versus only 23% for the published sequence. Dz858 recognized 16% of HCV type 4 versus 50% for the published sequence. The published sequence recognized nearly 90% of HCV type 5 and 6 versus less than 5% for Dz858.

Our Dz858 construct recognized at a 100% match level, sequences that did not match the published sequence.

A BLAST search using similar criteria as those described above, was conducted with our Dz348 construct and another sequence published in U.S. application Ser. No. 09/817,879 to Blatt et al. Results of the BLAST search indicate that Dz348 recognized 58% of all HCV genotypes recovered from the HCV database. However, the published sequence from Blatt et al., was 100% identical to less than 1% of the isolates found in the database. Results for individual genotypes from the pooled data indicate that Dz348 100%-matched over 90% of the HCV genotypes for type 1, 1b, 4 and 5 versus a 1% or less for the published sequence. BLAST results for HCV genotypes 2 and 5 indicate that Dz348 recognized 21% and 3% of HCV sequences respectively and that Blatt's published sequence did not match any. This indicates that Dz348 has a significant advantage in HCV genotype recognition.

A further BLAST search was conducted with our Dz699 construct and a further sequence published in U.S. application Ser. No. 09/817,879 to Blatt et al, using similar criteria as those described above. Results of this BLAST search indicate that overall, Dz699 recognized 49% of all HCV genotypes recovered from the HCV database versus 42% for Blatt's published sequence. Results for individual genotypes also varied. For example, Dz699 matched 50% of the HCV genotypes for types 1, 1b. The published sequence also matched 50% of other sequences among HCV genotypes for types 1, 1b. Dz699 matched 28% of HCV type 2 versus 66% match for the published sequence. Dz699 matched 73% of HCV type 3 versus 16% for Blatt's published sequence. Dz699 matched 67% and 60% of HCV types 5 and 6 respectively versus 17% and 30%, for Blatt's published sequence.

It would be advantageous to provide a pharmaceutical composition comprising at least two DNAzymes covering a similar region but having one nucleotide difference compared to one another. Such pharmaceutical composition may allow perfect match with more HCV isolates and/or genotypes and may thus provide better treatment and/or protection against HCV infection and HCV-related disease. The first DNAzyme may possess, for example, the exact sequence disclosed herein of an active fragment thereof (a fragment able to cleave HCV) and the second DNAzyme may possess one nucleotide (base) variation compared to the first DNAzyme. The nucleotide variation may be found, more particularly, in the annealing arm of the DNAzyme.

In accordance with the present invention the first and second (or more) DNAzymes may cover for example, a region as found in SEQ ID NO.:22 or 25 or a fragment thereof. The first DNAzyme may comprise for example, a sequence of at least 7, at least 8, at least 9 of to the last nucleotide of SEQ ID NO.:20 and a sequence of at least 7, at least 8, at least 9 of the first nucleotide of SEQ ID NO.:21 and the second DNAzyme may have a nucleotide variation compared to the first nucleotide. More particularly the first DNAzyme may have a first annealing arm complementary to at least 7, at least 8, at least 9 nucleotides on one side of the predicted cleavage site of SEQ ID NO.:22 or 25 and a second annealing arm complementary to at least 7, at least 8, at least 9 nucleotides on the other side of the predicted cleavage site of SEQ ID NO.:22 or 25. Again, the second DNAzyme may have at least one nucleotide variation compared to the first DNAzyme, but more specifically, one nucleotide variation.

A pharmaceutical composition which would comprise a third DNAzyme may be different, for example, from the first and second DNAzymes by one nucleotide variation.

Similar pharmaceutical composition comprising at least a first and second DNAzymes may be made for Dz348 and Dz699.

It is to be understood herein that the DNAzymes described herein may be slightly shorter than illustrated or may be slightly longer than illustrated. Shorter DNAzymes may comprise for example fragments of DNAzymes described herein, encompassing at least 7 nucleotides on each side of the predicted cleavage site. Longer DNAzymes may comprise, for example, the DNAzymes sequences described herein (including those with a nucleotide variation) and may also comprise one or more nucleotide on either or both sides complementary to the HCV genome (FIG. 2) or HCV isolates.

For example, as the homology in the region of the HCV genome targeted by Dz858-15-15 is sufficiently high among several HCV subtypes (one nucleotide variation), a drug which would comprise either one or the other of the nucleotide variations in this region is expected to have a catalytic activity against all of these subtypes. Alternatively, a drug which comprises a combination of at least two deoxyribozyme variants, each carrying the above mentioned nucleotide variation will therefore be efficacious against all of the 100 subtypes.

Using deoxyribozyme design methods similar to those outlined by Oketani et al [7] and the m-fold program, has enabled us to examine possible secondary structures contained within the first two-thirds of the HCV genome (bases 1 to 6000). We observed that all of the 30 possible secondary structures generated by the m-fold program permitted annealing of Dz858-15-15 to a single-stranded region in our HCV-1 b RNA sequence.

### Example 2

### Enzymatic Analysis of Phosphorothioate-Based Deoxyribozymes

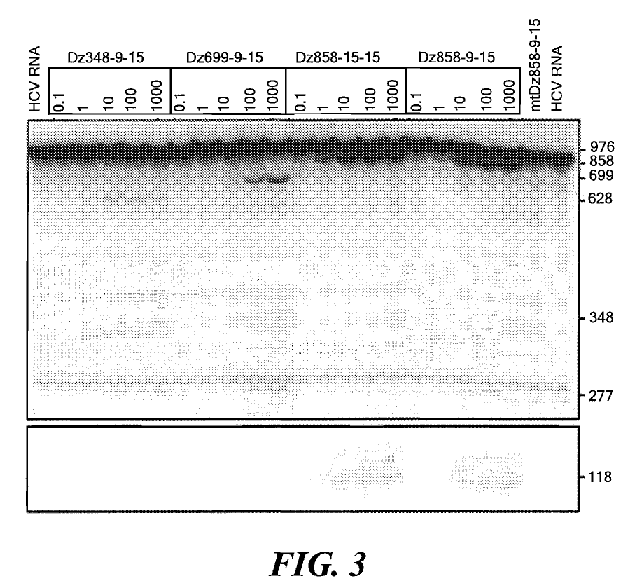

In vitro cleavage was performed using a radiolabeled synthetic HCV RNA produced in vitro using pGEM-7Zf-HCV DNA template and T7 RNA polymerase. The RNA substrate spanned HCV UTR-core genomic position 1 to 976. As shown in a representative urea-polyacrylamide gel illustrating deoxyribozyme cleavage activity for our deoxyribozyme series and for the mutated deoxyribozyme, mtDz858-9-15end (FIG. 3), we observed cleavage of HCV RNA by Dz348-9-15end into two fragments of 348 and 628 nucleotides. Dz699-9-15end cleaved HCV RNA into fragments of 699 and 277 nucleotides, while Dz858-9-15end gave fragments of 858 and 118 nucleotides. Based on predicted cleavage sites within the 976-base HCV RNA substrate, all three deoxyribozymes properly cut their HCV substrates into appropriate fragment lengths. Conversely, incubation of the HCV RNA substrate with mtDz858-9-15end using a substrate to enzyme (S:E) ratio up to 1:1000 resulted in no detectable cleavage activity (FIG. 3, lane mtDz858-9-15end). Similarly, mtDz348-9-15end and mtDz699-9-15end also failed to display significant cleavage activity when assayed with the HCV RNA substrate (S:E ratio of 1:1000, data not shown).

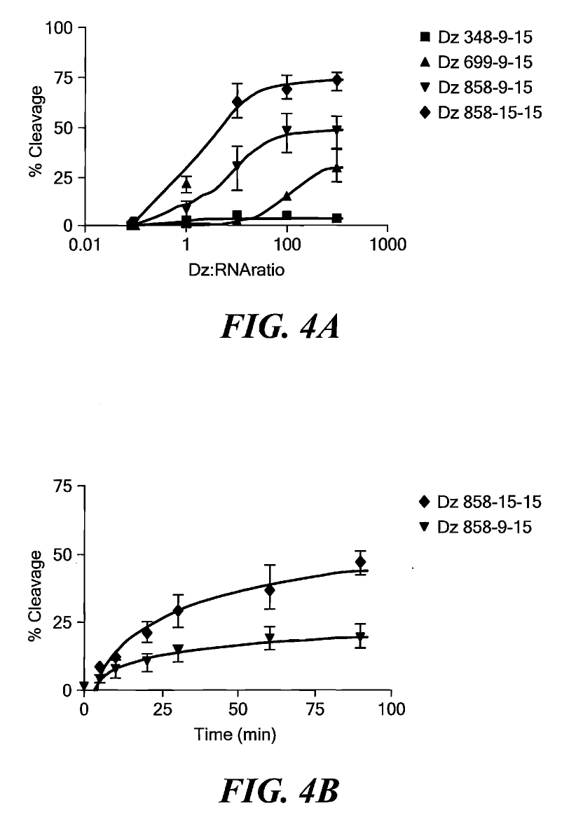

We have investigated whether increasing the deoxyribozyme arm length up to 15 nucleotides would augment deoxyribozyme catalytic efficiency [7, 24]. However, while increasing arm length may increase deoxyribozyme arm affinity, thereby decreasing Kₘ and augmenting the percent cleavage, this longer arm length may also affect the ability of the deoxyribozyme to release from its target thus lowering overall catalytic activity or K_{cat} [15]. We investigated whether increasing the arm length of our more active deoxyribozyme, namely Dz858-9-15end, from 9 to 15 residues would decrease Kₘ without compromising overall catalytic efficiency. Cleavage studies (n=3) similar to those illustrated in FIG. 3 revealed that lengthening the arm of Dz858-9-15end by six nucleotides (Dz858-15-15end) resulted in the highest cleavage efficiency (FIG. 4a and Table 2). Dz858-15-15end cleaved HCV RNA more efficiently than did Dz858-9-15end, which in turn cleaved HCV RNA more efficiently than Dz699-9-15end. Dz348-9-15end failed to exhibit significant cleavage activity above background levels (FIG. 4a). The structure of Dz348-9-15end therefore may require optimization.

However, it is also possible that Dz348-9-15end does not cut HCV mRNA as might be predicted by an open structure using the m-fold program, as the HCV sequence may not form an open structure and may be inaccessible for annealing to Dz348-9-15end. At the initial Vₘₐₓ plateau (i.e. S:E ratio of 1:100), we observed that Dz858-15-15end cut HCV RNA substrate 2.5-, 5.7- and 14-fold greater than Dz858-9-15end, Dz699-9-15end and Dz348-9-15end, respectively (FIG. 4a). As shown in FIG. 4a and Table 2, addition of six nucleotides to the 5′ arm of Dz858-9-15end, decreased Kₘ by 3-fold and increased catalysis by 50% (Table 2). The poor ability of Dz348-9-15end to cleave the RNA substrate (FIG. 4a, <5% for all concentrations tested) may be due to a higher preference for type II deoxyribozymes to cleave sequences containing an unpaired purine and a paired pyrimidine residue such as the AU or GU pairs found in Dz858-9-15end, Dz858-15-15end and Dz699-9-15end cleavage sites versus the AC pair found at the Dz348-9-15end HCV cleavage site. Another hypothesis for the poor cleavage is the potential masking of the Dz348-9-15 cleavage site by HCV RNA secondary structure [15, 28].

The increase in Dz858-15-15end enzyme activity versus that for Dz858-9-15end was also apparent when incubation times were varied. The enzymatic activity for Dz858-9-15end was shown to plateau at 60 minutes, while that for Dz858-15-15end levelled off at 90 minutes, and Dz858-15-15end achieved a 2.5-fold greater level of cleavage product compared to Dz858-9-15end (FIG. 4b).

Based on the above experiments, we observed that increasing the 5′ arm length of Dz858-9-15end from 9 to 15 nucleotides (designated Dz858-15-15end) increased the cleavage efficiency four-fold when measured in an in vitro cleavage assay (Table 2, Kcat/Km). The increase in Dz858-15-15end cleavage efficiency appeared in part due to a 3-fold decrease in Km and a small increase in Kcat. These findings were consistent with observations seen by other investigators who also noted an increase in cleavage activity upon lengthening the deoxyribozyme recognition arms [7, 24]. However, it was surprisingly found herein that increasing the arm length did not impair the overall efficiency of the DNAzymes.

TABLE 2
Deoxyribozyme binding and catalytic constants

| Deoxyribozyme | Km_{(mol/L)} | Kcat₍ₘᵢₙ₋₁₎ | Kcat/Km_{(mol/L)-1 min-1} |
|---|---|---|---|
| Dz699-9-15 | 1.5 × 10⁻⁵ | 5.9 × 10⁻³ | 4.0 × 10² |
| Dz858-9-15 | 5.8 × 10⁻⁷ | 8.0 × 10⁻³ | 1.4 × 10⁴ |
| Dz858-15-15 | 2.1 × 10⁻⁷ | 1.2 × 10⁻² | 5.7 × 10⁴ |

Deoxyribozyme, Dz
Dz affinity for RNA target, Km.
Maximum catalysis for RNA target, Kcat.
Dz catalytic efficiency, Kcat/Km.

### Example 3

### Intracellular Activity of Phosphorothioate-Based Deoxyribozymes

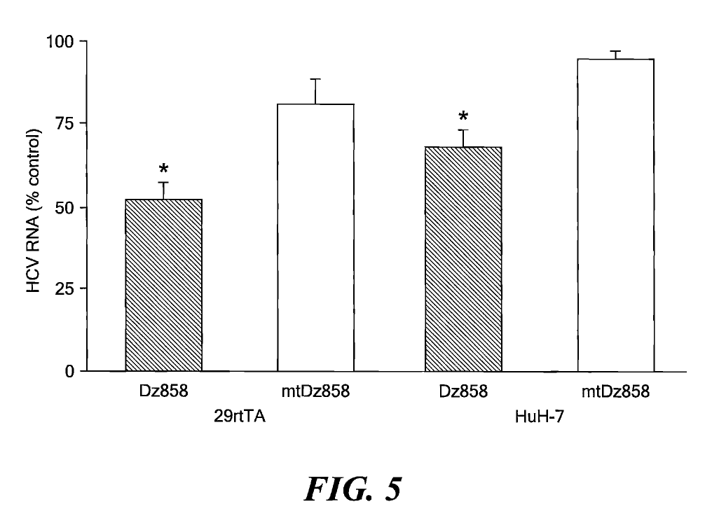

While Dz858-15-15end was capable of recognizing and efficiently cleaving HCV RNA in vitro, an earlier report by Oketani et al cautioned that although a given deoxyribozyme would exhibit high K_{cat}/Kₘ values in vitro, this same molecule might cleave poorly when tested against its target RNA inside living cells [7]. Therefore we tested whether HCV RNA target expressed within cells would be accessible for Dz858-15-15end hybridization and cleavage. Dz858-15-15end was co-transfected with the HCV RNA expression plasmid. For experimental purposes we chose as hosts the highly transfectable human epithelial cell line 293rtTA, which is capable of generating a high level of expression plasmid RNA transcripts, and the HCV permissive hepatoma cell line HuH-7. As shown in FIG. 5, HCV RNA signal was greatly reduced in 293rtTA cells treated with Dz858-15-15end but not mtDz858-15-15end. Results from three transfection experiments indicated that HCV RNA was reduced by 48%±5 SEM (p=0.004, HCV RNA versus HCV RNA+Dz858-15-15end) (FIG. 5). This reduction in HCV RNA appeared not to be due simply to antisense annealing by the deoxyribozyme to its RNA target [7], as mtDz858-15-15end, for which the catalytic domain alone was altered, exhibited only a 19%±8 SEM reduction in HCV RNA signal (p=0.3, HCV RNA versus HCV RNA+mtDz858-15-15end) (FIG. 5).

Testing of Dz858-15-15end in the HCV host cell line HuH-7 (n=3) further confirmed that Dz858-15-15end was capable of reducing intracellular HCV RNAs (FIG. 5). Dz858-15-15end reduced HCV RNA in HuH-7 cells by 32%±6 SEM (p=0.02, HCV RNA versus HCV RNA+Dz858-15-15end), whereas mtDz858-15-15end reduced HCV RNA by only 6%±2 SEM (p=0.2, HCV RNA versus HCV RNA+mtDz858-15-15end) (FIG. 5). Thus our intracellular studies indicate that Dz858-15-15end is capable of recognizing and cutting intracellular HCV RNA in two cell models.

Therefore, under simulated physiological conditions described above, Dz858-15-15end achieved maximal intracellular HCV RNA reductions of 32% and 48% in hepatoma and epithelial cells, respectively. Our inability to attain complete intracellular cleavage and an observed variance in intracellular cleavage between HuH-7 and 293rtTA suggests that Dz858-15-15end may have been sequestered to an unproductive intracellular location possibly bound to intracellular proteins via the phosphorothioate residues, or it may have encountered interference in its recognition of the HCV RNA target [38-41]. Therefore, we may not have achieved the maximum potential of the capacity of intracellular cleavage attainable in our two assay systems. Improvement in RNA cleavage upon application of newer nucleotide designs or the employment of alternative means for the introduction of deoxyribozymes into hepatocytes [39,42,43] is therefore further investigated.

Dz858-15-15end displayed enzymatic activities comparable to other therapeutically valuable deoxyribozyme targets [15, 16, 34] and was equal to or slightly superior to deoxyribozymes that have been reported to cleave intracellular HCV RNAs [7], as noted by a 32% to 48% reduction in HCV RNA after 24 hours of deoxyribozyme exposure for human hepatoma and epithelial cells, respectively [7].

### Example 4

### Generation of O-Methyl Deoxyribozyme Variants

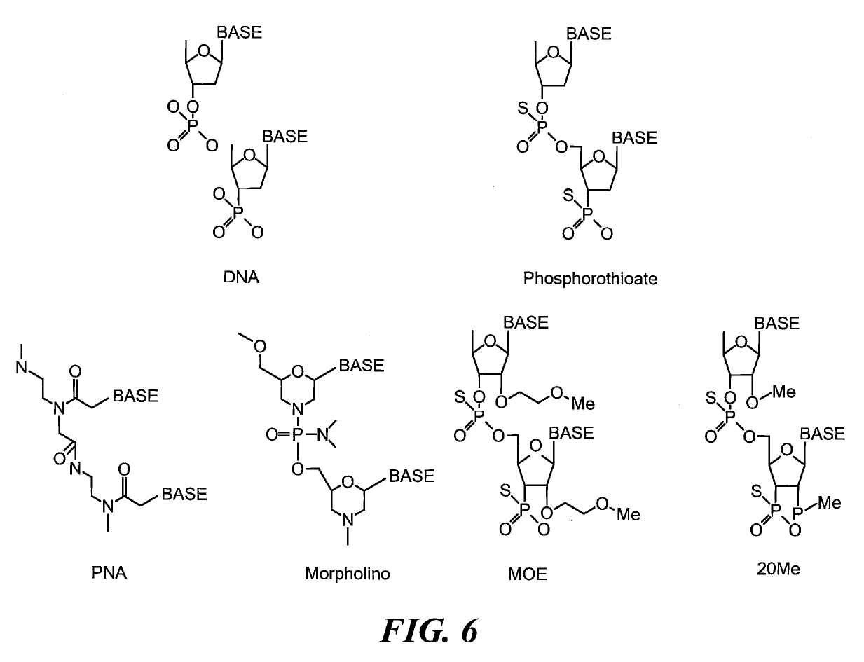

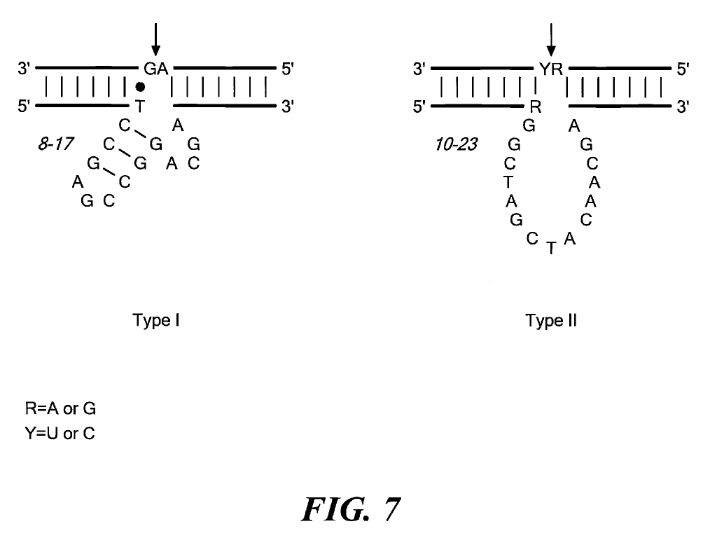

Our current drug candidate, Dz858-15-15end, utilizes two phosphorothioate-linked nucleotides in each of the flanking arms (2 residues/arm end) (FIG. 6, FIG. 1A). Although favorable pharmacokinetics and therapeutic outcomes for phosphorothioate-based oligonucleotides have made them the dominant platform for various nucleic acid-based therapies and were important criteria in the original design of our deoxyribozyme library, phosphorothioate-based oligonucleotides may stick to a wide variety of serum and cellular proteins. This may result in decreased pK profiles or cause a reduction in the effective drug dose. Additionally, phosphorothioate-protein interactions may result in complement activation, thrombocytopenia and mild acute-phase responses leading to increased patient morbidity.

Therefore, deoxyribozyme variants are generated to lessen or eliminate these potential issues. Nucleotide substitutions are therefore introduced in Dz858-15-15 to improve its biological stability and in vitro efficacy profile, eliminate possible phosphorothioate-related cytotoxicity, and enhance its pharmacokinetic and efficacy profiles.

Type II deoxyribozyme variants were synthesized using phosphoramidite chemistry (integrated DNA Technologies, Coralville, Iowa) and subsequently purified by salt exchange.

To generate sufficient HCV RNA target substrate the cDNA sequence from pGEM-7Zf-HCV spanning the HCV 5′UTR and the adjoining core protein coding sequence (HCV genome positions 1 to 976) was amplified by polymerase chain reaction (PCR) using the sense primer 5′-TGTAATACGACTCACTATAGCGA-3′ (SEQ ID NO.:4) encoding the bacteriophage T7 RNA polymerase promoter and an anti-sense HCV-encoding primer 5′-TCATACACAATGCTTGCGTTG-3′ (SEQ ID NO.:5) as described herein. Amplified DNA was fractionated by agarose gel electrophoresis and purified using the QIAquick gel extraction kit (Qiagen Inc., Mississauga, ON). Radiolabeled RNA substrate was generated as recommended by the manufacturer, using 1 μg amplified HCV cDNA, the MegaScript T7 transcription kit (Ambion Inc., Austin, Tex.), T7 RNA polymerase (Ambion Inc.) and [³²P]UTP (20 mCi/ml, 800 Ci/mmole) (Amersham, Piscataway, N.J.). Transcription reactions were performed at 37° C. for 6 h followed by DNA template removal using RNAse-free DNAse (Ambion Inc.), phenol-chloroform extraction and ethanol precipitation.

### Example 5

### Enzymatic Analysis of O-Methyl Deoxyribozyme Variants

100 nM radiolabeled HCV target RNA substrate was suspended in 50 mM Tris-HCl buffer pH 7.5 containing 10 mM MgCl₂ and incubated for 1 h at 37° C. with increasing log₁₀ concentrations of deoxyribozyme. Cleavage reactions were terminated by the addition of gel loading buffer containing 95% formamide, and RNA cleavage products resolved by gel electrophoresis in a 6% polyacrylamide gel containing 8 M urea and Tris-borate buffer. Following electrophoresis, gels were dried and cleavage products quantified using the SI Phosphorlmager (Molecular Dynamics, Sunnyvale, Calif.). Relative band intensity for the cleavage products was plotted as the percentage of cleaved RNA versus the deoxyribozyme concentration.

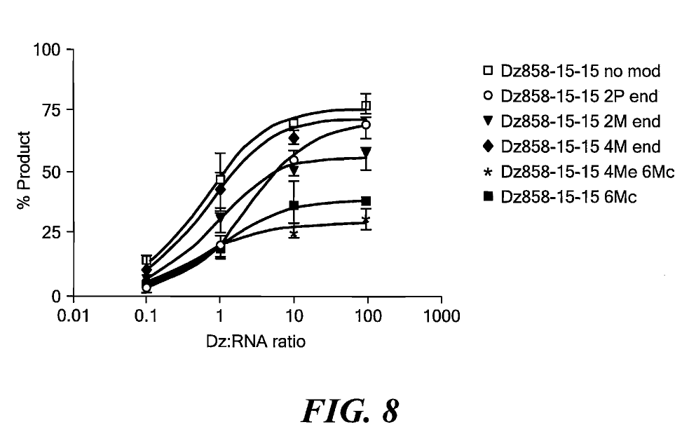

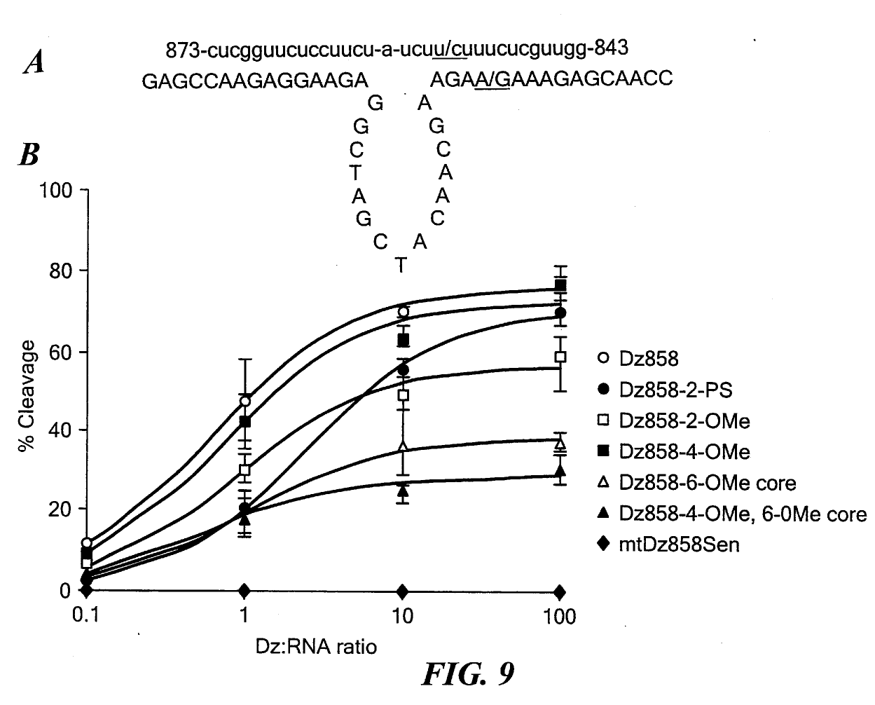

Results of in vitro cleavage experiments using concentrations of deoxyribozyme higher than 23 nM (for eg. 600 nM) indicate that the highest percentage of product cleavage is obtained with the unmodified Dz858-15-15 (FIG. 8, open square). Unmodified deoxyribozymes, however, rapidly degrade in the presence of nucleases under physiological conditions. Therefore unmodified Dz858-15-15 was modified by addition of phosphorothioate nucleotides or nucleotides containing a methyl group at the 2′ OH position of the furan ring. Although the phosphorothioate is expected to increase the half-life of the Dz, the cleavage efficiency of Dz858-15-15end containing two phosphorothioate nucleotide additions located at each of the two ends (FIG. 8, open circle) was less compared to unmodified Dz858-15-15. Addition of four nucleotides containing 2′-O-methyl additions at each of the two end nucleotides of the Dz858-15-15 deoxyribozyme (FIG. 8, filled diamond) was highly comparable to unmodified Dz858-15-15 in the ability to cleave HCV target RNA (FIG. 8, open square versus filled diamond, respectively). However, the 2′-O-methyl modification is expected to have an increased half life on the order of 10-fold compared to unmodified deoxyribozyme when placed into human eukaryotic cells. Results of in vitro cleavage experiments using concentrations of deoxyribozyme of about 23 nM (shown in FIG. 9) show that the enzymatic activity of the Dz858-15-15 deoxyribozyme variant containing four 2′-O-methyl additions was comparable to unmodified Dz858-15-15 deoxyribozyme. FIG. 9 also shows that incubation of radiolabeled HCV RNA target with 1000-fold molar excess of mutant (mt) Dz858-15-15 revealed no detectable cleavage. This data indicated that Dz858-15-15 4M-end was superior in the ability to cleave HCV RNA target.

As shown in Table 3, comparison of cleavage activity among Dz858 variants containing different nucleotide modifications revealed that the Dz858 variant with four OMe additions on each annealing arm exhibited a four-fold increased enzymatic activity compared to Dz858 homologues possessing two PS modifications (2.07×10⁵ Kcat/Km for Dz858-15-15 4M-end versus 5.66×10⁴ Kcat/Km for Dz858-15-15 2P-end (Table 3). Dz858-15-15 4M-end enzymatic activity was comparable to unmodified Dz858 (2.42×10⁵ Kcat/Km, Table 3). Unmodified Dz858-15-15 demonstrated a Kcat/Km of 2.42×10⁵ which differed from Dz858-15-15 4M-end by less than 14% (Table 3). Other DNAzymes having modified nucleotides were tested.

TABLE 3
List of Dz858-15-15 modifications
and respective enzyme efficiencies

| | Km | Kcat | Kcat/Km |
|---|---|---|---|
| DNAzyme | (mol/L) | (min − 1) | (mol/L) − 1 min − 1 |
| Dz858-15-15 | 4.86 × 10⁻⁸ | 1.17 × 10⁻² | 2.42 × 10⁵ |
| unmodified DNA | | | |
| Dz858-15-15 | 2.13 × 10⁻⁷ | 1.20 × 10⁻² | 5.66 × 10⁴ |
| 2P-end | | | |
| Dz858-15-15 | 8.62 × 10⁻⁸ | 9.39 × 10⁻³ | 1.09 × 10⁵ |
| 2M-end | | | |
| Dz858-15-15 | 5.291 × 10⁻⁸ | 1.10 × 10⁻² | 2.07 × 10⁵ |
| 4M-end | | | |
| Dz858-15-15 | 5.84 × 10⁻⁸ | 4.94 × 10⁻³ | 8.45 × 10⁴ |
| 4M-end, 6M-core | | | |
| Dz858-15-15 | 5.78 × 10⁻⁸ | 5.36 × 10⁻³ | 9.28 × 10⁴ |
| 6M-core | | | |

Dz, Deoxyribozyme
P, phosphorothioate
M, 2′-O-methyl

### Example 6

### Demonstration of In Vitro Efficacy of Dz858-15-15 Using Phosphorothioate-Modified Dz858-15-15 and 2′-O-methyl-Modified Dz858-15-15

### Synthesis of Genomic-Length HCV RNA

Genomic-length HCV RNA deoxyribozyme substrate was generated from 1 μg of plasmid pGEM-7Zf-HCV (kindly provided by Dr S. Mounir, Shire Pharmaceuticals, Laval, QC).

The plasmid portion of pGEM-7Zf-HCV was derived from the parental vector pGEM-7Zf (Promega, Madison, Wis., EMBL accession Nos. X65310, X6531 1) to which the 9.6 kilobase cDNA encoding full-length genome of HCV type 1b (NCBI accession No. M58335, NID g329770) flanked by HindIII and XbaI DNA restriction enzyme recognition sequence was inserted at the HindIII and XbaI restriction site of the pGEM-7Zf vector.

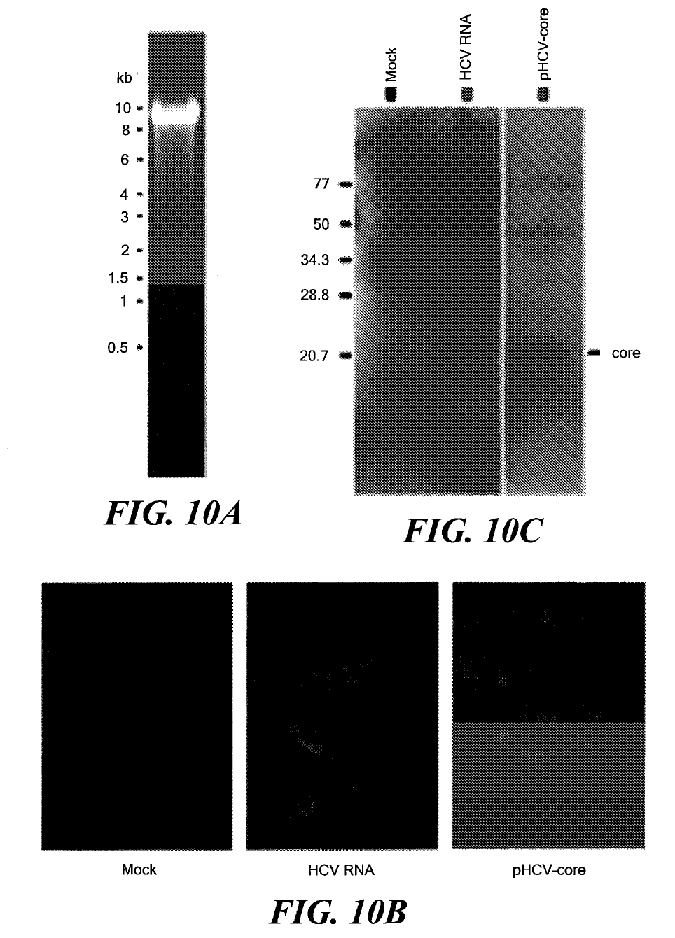

pGEM-7Zf-HCV thus contains the T7 RNA polymerase recognition sequence and the cDNA sequence for an entire HCV Type 1 genome sequence [21] flanked on the left end by restriction site Hind III and on the right end by restriction site Xba I. Genomic length HCV RNA was generated following XbaI digestion of pGEM-7Zf-HCV, and then using MegaScript T7 transcription kit (Ambion Inc., Austin, Tex.) and T7 RNA polymerase (Ambion Inc.). Transcription reactions were performed at 37° C. for 6 h followed by the removal of plasmid DNA template using RNAse-free DNAse (Ambion Inc.). The synthetically-produced RNA was phenol-chloroform extracted, ethanol precipitated and suspended in RNAse free water to a final concentration of 2.6 μg/μl. HCV RNA was electrophoretically resolved in a 0.8% formaldehyde-agarose gel and was noted to migrate at the expected range of <9 kilobases. This <9 kilobase RNA is in agreement with the expected size of 9.4 kilobases for the full-length genomic HCV RNA (FIG. 10A) [21].

### Validation of HCV Core Expression

The human hepatoma cell line HuH-7 containing the neomycin (neo) resistance gene, kindly provided by Dr Tatsuo Takahashi (Health Science Research Resources Bank, Japan) was cultured in Dulbecco's Modified Eagles Medium (DMEM) (Invitrogen Inc., Burlington, ON) supplemented with 10% fetal bovine serum (Medicorp Inc., Montreal, Qc) and antibiotics. Full-length HCV RNA was transfected into HuH-7 cells. This synthetic genome-length RNA is predicted to locate in the cytoplasm and fold into a functionally active form. An eukaryotic expression plasmid pHCV-core (J. E. Tanner, unpublished) was used to validate HCV core protein expression in this HCV liver cell model. Briefly, 3×10⁴ Huh-7 cells were transfected with either 325 ng HCV RNA or pHCV-core using 1 μg Lipofectamine 2000. Cells were cultured for 24 h on glass slides and processed for immunofluorescence using pooled HCV-positive human serum, followed by biotinylated goat (Fab)2 anti-human IgG (1:600, Jackson ImmunoResearch Laboratories, West Grove, Pa.) and 3 μg/ml dichlorotriazinyl amino fluorescein (DTAF)-conjugated streptavidin (Jackson ImmunoResearch). The cell extracts were also processed for SDS-PAGE and immunoblofted with HCV-positive human serum (1:500), biotinylated-goat (Fab)2 anti-human IgG (1:8000), followed by horseradish peroxidase (HRP)-streptavidin (1:6000, Sigma-Aldrich, Oakville, ON) and chemiluminescence substrate (Sigma-Aldrich). As shown in FIG. 10 panels B and C, HCV protein expression, including core protein, was detected in Huh-7 cells transfected with genomic-length RNA. The expression of HCV core protein was comparable to that seen in Huh-7 cells transfected with pHCV-core (FIG. 10 panels B and C, pHCV-core). This indicated that a synthetic genomic-length HCV RNA could properly locate to the cytoplasm and fold into a functionally active form capable of RNA translation initiation from the HCV IRES. This also suggest that a full-length synthetic HCV RNA may reliably serve as a Dz target, comparable to endogenous HCV RNA.

### In Vitro Efficacy

HuH-7 cells were first seeded at 2.4×10⁵ cells per well in 12-well tissue culture plates and grown overnight. The HuH-7 cells were then co-transfected in a final volume of 200 μl of Opti-MEM containing 1 μg of our synthetically-produced genomic-length HCV RNA and 600 nM of phosphorothioate-modified or 2′-O-methyl-modified Dz858-15-15 using 3 g of Lipofectamine 2000 (Invitrogen Inc.) (See Table 4). In experiments outlined in Table 5, Dz-858-15-15 was transfected into HuH-7 cells 6 hours prior to the transfection of HCV genomic-length RNA. After 6 hours, the medium was removed and replaced by fresh DMEM medium supplemented with 10% fetal bovine serum. Following 24 hours of culture, the total cellular RNA was extracted using TRI Reagent (Molecular Research Center, Cincinnati, Ohio) and resuspended in 25 μl of RNAse-free water.

The amount of cellular HCV genomic RNA and the amount of cellular neomycin resistance gene were determined by reverse transcriptase quantitative polymerase chain reaction (RT-qPCR) analysis using HCV sense primer 5′-GGCGTGAACTATGCAACAGGGAAT-3′ (SEQ ID NO.:58), 6′-carboxyfluorescein, succinimidyl ester (FAM)-labelled HCV probe, 5′-TTCCGCTTACGAAGTGCACAACGTGT-3′ (SEQ ID NO.:59) and HCV antisense primer 5′-TGGAGCAGTCGTTCGTGACATGAT-3′ (SEQ ID NO.: 60), or neo sense primer 5′-ACCTTGCTCCTGCCGAGAAAGTAT-3′ (SEQ ID NO.:11), 6′-carboxy-2′,4,4′,5′,7,7′-hexachlorofluorescein, succinimidyl ester (HEX)-labeled neo probe 5′-AATGCGGCGGCTGCATACGCTTGAT-3′ (SEQ ID NO.:61) and neo antisense primer 5′-CGATGTTTCGCTTGGTGGTCGAAT-3′ (SEQ ID NO.:13; synthesized by Integrated DNA Technologies Inc., Coralville, Iowa) in conjunction with the QuantiTect Multiplex RT-PCR Kit (Qiagen, Mississauga, ON). The RT step was performed for 30 minutes followed by Taq activation by incubation at 95° C. for 10 minutes. PCR was performed by heating the sample for 15 minutes at 95° C., and 45 amplification cycles. Each amplification cycle consisted of a 45 seconds incubation at 95° C. and a 45 second annealing and elongation step at 60° C. in a Mx3000P real-time PCR thermocycler (Stratagene Inc. La Jolla, Calif.). Logarithmic concentrations of pHCV-UTR-core containing HCV core sequences as well as the neomycin resistance gene served as DNA reference standards during RT-qPCR analysis [21].

TABLE 4
Percent reduction in HCV RNA signal in HuH-7 cells when
Dz858-15-15 is co-transfected with HCV genomic RNA

| Dz modification | Exp. 1 | Exp. 2 | Exp. 3 | Average |
|---|---|---|---|---|
| Phosphorothioate-modified | 80.8 | 71.8 | 77.1 | 76.6 ± 2.6 |
| Dz858-15-15 | | | | |
| 2′-O-methyl-modifed | 85.2 | 81.4 | 83.4 | 83 ± 1.1 |
| Dz858-15-15 | | | | |

TABLE 5
Percent reduction in HCV RNA signal in HuH-7
cells following transfection of Dz858-15-15
followed by transfection of HCV genomic RNA

| Dz modification | Exp. 1 | Exp. 2 | Exp. 3 | Average |
|---|---|---|---|---|
| Phosphorothioate-modified | 51 | 34.8 | 33.47 | 39.8 ± 9.8 |
| Dz858-15-15 | | | | |
| 2′-O-methyl-modifed | 67.5 | 46.5 | 13.5 | 42.5 ± 13.5 |
| Dz858-15-15 | | | | |

Results of these experiments indicate that phosphorothioate-modified or 2′-O-methyl-modified Dz-858-15-15 reduced HCV RNA cellular levels by 76.6%±2.6 SEM and 83%±1.1 SEM, respectively (Table 4). Reduction in HCV RNA following exposure of HuH-7 cells to phosphorothioate-modified or 2′-O-methyl-modified Dz-858-15-15 was statistically significant (p-value=0.0012 and p-value=0.0002, respectively) using the “t-test for hypothesis of the mean” and a significance level of α=0.05. Comparison of the HCV RNA signal reduction following exposure to phosphorothioate-modified versus 2′-O-methyl-modified Dz-858-15-15 indicated that 2′-O-methyl-modified Dz858-15-15 reduced HCV RNA 7% more than phosphorothioate-modified Dz858-15-15 (p-value of 0.075 using the “t-test for differences in two means” and a level of significance of α=0.10). This demonstrates that 2′-O-methyl-modified Dz-858-15-15 has increased in vitro efficacy in HuH-7 cells as compared to phosphorothioate-modified Dz858-15-15.

Results also indicate that pre-exposure of HuH-7 cells to phosphorothioate-modified or 2′-O-methyl-modified Dz-858-15-15 prior to the introduction of HCV genomic RNA reduced HCV cellular levels by 39.8%±9.8 SEM and 42.5%±27 SEM, respectively (Table 5). Reduction in HCV RNA following exposure of HuH-7 cells to phosphorothioate-modified or 2′-O-methyl-modified Dz-858-15-15 was statistically significant (p-value=0.008 using the “t-test for hypothesis of the mean” and a level of significance of α=0.05) for phosphorothioate-Dz858-15-15 and near-statistically significant for 2′-O-methyl-modified Dz858-15-15 (p-value=0.06).

### Example 7

### Contrasting In Vitro Efficacy of Various Forms of Dz858-15-15 Prior to, Concomitant with or Following Genomic Length HCV RNA Transfection

Dz858-15-15 4M-end, along with a sense-mutant form of Dz858 (mtDz858Sen; GGTTGCTCTTTTTCTGGCGAGCTACAACGATCTTCCTCTTGGCTC SEQ ID NO.:), Dz858 Dz858-15-15 2P-end, Dz2 (7) (GSCSACGGTCTACGAGAGGCTAGCTACAACGACTCCCGGGGCACTSCSG SEQ ID NO.:) and an OMe-modified form of Dz2 (Dz2-4-Ome; MGMCMAMCGGTCTACGAGAGGCTAGCTACMCGACTCCCGGGGCAMCMTMCMG SEQ ID NO.: ) containing OMe additions identical to that of Dz858-15-15 4M-end were tested for their ability to cleave genomic-length HCV RNA contained within Huh-7 cells. Dz2 and its OMe variant were included for comparison as Dz2 was previously shown to cleave the subgenomic HCV RNA target containing the HCV 5′UTR-core protein coding sequence (7; 47). All Dz were added 6h prior to (pre-addition), concomitant with (co-addition) or 6h following (post-addition) HCV RNA transfection. Briefly, 1 μg synthetic HCV RNA was transfected into Huh-7 cells followed by addition of 23 nM of individual Dz using 3 μg Lipofectamine. The 23 nM Dz concentration (HCV RNA molar ratio of 75:1) was previously found to cleave intracellular genomic HCV RNA when compared to the sense-catalytic-ablated Dz, mtDz858Sen, and to exhibit no overt toxicity (J. B. Trepanier, unpublished). Changes in HCV RNA concentration were measured by RT-qPCR in conjunction with the QuantiTect Multiplex RT-PCR Kit (Qiagen, Mississauga, ON) as previously presented. β-actin RNA served as a measure of total RNA concentration and was determined using β-actin sense primer 5′-CCTTCCTGGGCATGGAGTCCT-3′ (SEQ ID NO.:) and antisense primer 5′-GGAGCAATGATCTTGATCTTC-3′ (SEQ ID NO.:) in conjunction with the QuantiTect SYBR Green RT-PCR Kit (Qiagen). Genomic-length HCV RNA was found to have an intracellular half-life of approximately 9 h (J. B. Trepanier, data not shown). Results are summarized in Table 6.

TABLE 6

% Reduction

A) Average % reduction in HCV RNA signal in HuH-7 cells
when Dz858-15-15 (23 nM) is pre-added with HCV genomic RNA

| | |
|---|---|
| mtDz858Sen | 0 ± 0 |
| Dz858-2-PS | 9.4 ± 9.4 |
| Dz2 | 11.1 ± 11.1 |
| Dz2-4-Ome | 33.6 ± 4.9 |
| Dz858-15-15-4M end | 62.5 ± 6.3 |

B) Average % reduction in HCV RNA signal in HuH-7 cells
when Dz858-15-15 (23 nM) is co-added with HCV genomic RNA

| | |
|---|---|
| mtDz858Sen | 0 ± 0 |
| Dz858-2-PS | 15 ± 7.7 |
| Dz2 | 0 ± 0 |
| Dz2-4-Ome | 9.2 ± 9.2 |
| Dz858-15-15-4M end | 64.8 ± 4.3 |

C) Average % reduction in HCV RNA signal in HuH-7 cells
when Dz858-15-15 (23 nM) is post-added with HCV genomic RNA

| | |
|---|---|
| mtDz858Sen | 0 ± 0 |
| Dz858-2-PS | 0 ± 0 |
| Dz2 | 0 ± 0 |
| Dz2-4-Ome | 12.2 ± 5.4 |
| Dz858-15-15-4M end | 60.4 ± 6 |

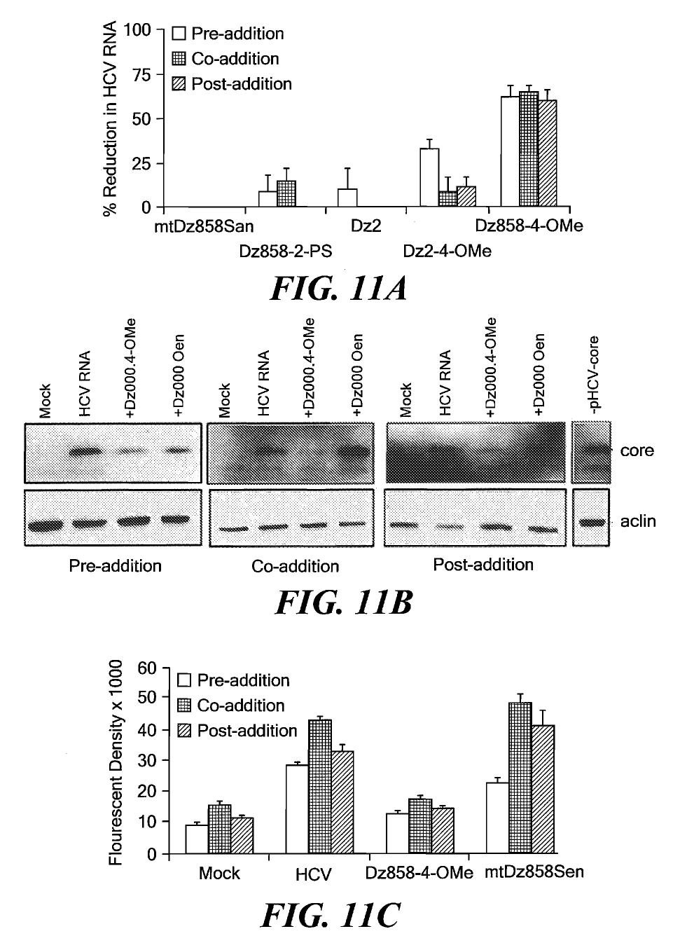

As shown in FIG. 11, when HCV genomic RNA was challenged with deoxyribozymes, results indicated that Dz858-15-15 4M-end reduced HCV RNA overall by 63%+2.7 SEM (FIG. 11A). mtDz858Sen displayed no HCV RNA cleavage, whereas Dz858-15-15 2P-end, Dz2 and Dz858-15-15 4M-end yielded an overall average HCV RNA reduction of 8.1%+2.9 SEM, 3.7%+3.7 and 18.3%+1.7, respectively, when tested in the three different drug addition protocols (FIG. 11A). The mean inhibition of Dz858-15-15 4M-end was highly significant when compared to Dz2, Dz2-4-OMe or Dz858-15-15 2P-end (p-values <0.001). We also noted that Dz858-15-15 4M-end reduced the expression of HCV core protein in Huh-7 cells in each of the three different drug administration regimes as measured by immunoblot and after normalization with parallel immunoblots containing cellular actin (FIG. 11B). Dz858-15-15 4M-end reduced HCV core protein by 55.2%, 96.6% and 87.3%, respectively, when added 6 h prior to, concurrently with, or 6 h after HCV RNA transfection (FIG. 11B). By comparison, mtDz858Sen reduced HCV core protein by 40.1%, 0% and 28.0%, respectively, for the three administration regimes cited (FIG. 11B). Overall, Dz858-15-15 4M-end reduced HCV core protein by 79.7%+12.6 SEM versus 22.7%+12.1 SEM for mtDz858Sen (p=0.032). In parallel experiments, further comparison of HCV core protein in Huh-7 cells was performed after deoxyribozyme treatment, immunostaining with pooled HCV-positive human serum and digital quantification of cellular immunofluorescence on over 100 cells present in eight to 10 independent microscopic fields (ImageJ software, Rasband, W.S., U.S. National Institutes of Health, Bethesda, Md., USA). Results indicate that Dz858-15-15 4M-end markedly reduced HCV immunofluorescence by an average of 86.1%+3.5 SEM for the three drug administration regimes (FIG. 11C). By comparison, mtDz858Sen failed to reduce HCV immunofluorescence in co-addition or post-addition experiments, and reduced HCV immunofluorescence by 28.8% in pre-addition experiments, yielding an overall reduction of 9.6%+9.6 (p=0.0017) (FIG. 11C). Comparison of Dz858-15-15 4M-end to a similarly modified variant of Dz2 (Dz2-4-OMe) revealed that addition of O-Me to a deoxyribozyme does not guarantee an increase in its cleavage efficiency. 4-OMe addition to Dz2 increased the cleavage of full-length HCV RNA by 14.6%. This would suggest that OMe interaction with an amenable nucleotide sequence such as Dz858 can be synergistic. Dz858-15-15 4M-end HCV core protein expression reduction, agrees favorably with the reduction in HCV RNA. Finally, the concentration of Dz858-15-15 4M-end (23 nM) needed to achieve a reduction in intracellular HCV RNA and protein target is far lower than that reported for other deoxyribozymes, ribozymes and antisense oligonucleotides whose effective doses ranged from 750 to 1000 nM and compared favorably to siRNA whose effective dose ranged from 25 to 100 nM (Seo, M. Y. et al. 2003 J Virol 77:810-812). All in all, Dz858-15-15 4M-end exhibited a 63%+2.7 SEM reduction of intracellular HCV RNA when tested in three different drug administrations, compared to Dz858-15-15 2P-end, Dz2 and Dz2-OMe which displayed an average of 8.1%+2.9 SEM, 3.7%+3.7 and 18.3%+1.7 reduction in HCV RNA, respectively. Interestingly, Dz858-15-15 2P-end and Dz2 were previously shown to reduce subgenomic HCV RNA targets in Huh-7 cells by 32% and 55%, respectively (7, 48).

### Example 8

### Generation of Morpholino Deoxyribozyme Variants

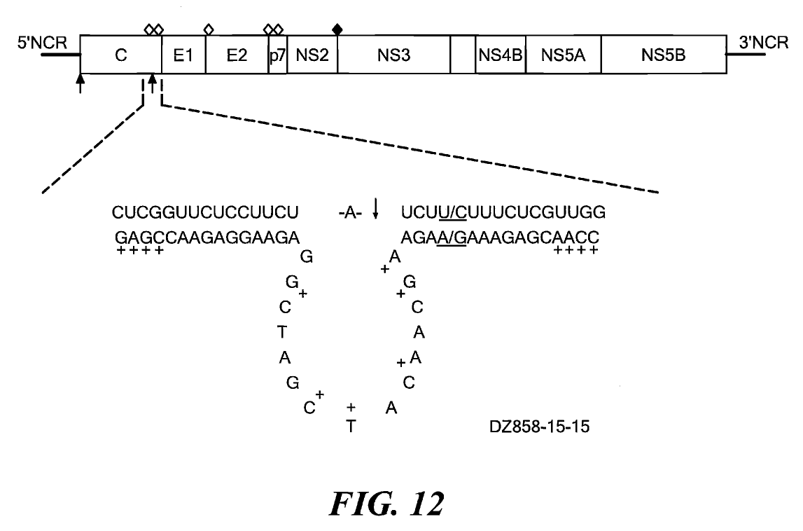

Based on the favorable preclinical findings for Dz858-15-15end and Dz858-15-15 4M-end, improved Dz858-15-15 variants are developed (FIG. 12). These variants use newly available and less toxic morpholino nucleotides for improving the in vitro and in vivo efficacy, stability and pharmaco-characteristics of the deoxyribozymes as well as a reduction in their cytotoxic properties.

Deoxyribozyme pharmacokinetics (pK), biodistribution in animals, potential toxicological properties and, most importantly, in vivo efficacy, are therefore explored for the deoxyribozyme variants of the present invention.

In general, when compared to other nucleotide designs or even in comparison to the newer small interference (si)RNAs, morpholino-based oligonucleotides (MBO) provide superior biostability, increased resistance to nuclease, better efficacy profiles, long-term biological activity and high aqueous solubility. MBOs also exhibit high target specificity and low protein interaction, and therefore may give reduced toxicity profiles. Further, MBOs are able to more readily ingress DNA and RNA secondary structures compared to other nucleotide formats. Because HCV RNA secondary structures are known to obstruct antisense DNAs, employment of MBO deoxyribozymes may improve recognition and annealing of Dz858-15-15 to its RNA target, yielding higher rates of HCV RNA cleavage. Further, MBOs have a long biological half-life, persisting up to seven days inside cells and at least 3 to 7 days in vivo, which allows for a lower dose of deoxyribozyme to yield superior in vivo efficacy profiles. Additionally, the MBO backbone exhibits minimal protein interaction and, therefore, low toxicity.

The morpholino deoxyribozymes are synthesized by Gene Tool LLC (Philomath, Oreg.). The morpholino-based Dz858-15-15 and its mutated counterpart, mtDz858-15-15 (underlined nucleotide) (FIG. 12) have the same sequence as the unmodified Dz858-15-15 and its mutated counterpart respectively, except that the former pair comprise some morpholino-based-nucleotides:

Dz858-15-15 (SEQ ID NO.:62):
5′-GAGCCAAGAGGAAGAGGCTAGCTACAACGAAGAA/GAAAGAGCAA
CC-3′
mtDz858-15-15 (SEQ ID NO.:63):
5′-GAGCCAAGAGGAAGAGGCGAGCTACAACGAAGAA/GAAAGAGCAA
CC-3′.

Therefore, the specificity of the morpholino-based DZ858-15-15 is not affected. Morpholino-based nucleotide derivatives are introduced at one or more of the positions (indicated by a + symbol FIG. 12) within the enzyme core as depicted by the loop structure and/or one or more positions within the two arms as depicted by the linear nucleotide sequences. The modification may be symmetrical or asymmetrical.

As indicated for the wild type Dz858-15-15 deoxyribozyme, the annealing arm sequence covers ˜36% of reported HCV core sequences. By introducing a single base change (i.e. A→G, shown in bold) into the Dz858-15-15 sequence, this ensures coverage of the remaining 64% of reported HCV sequences.

### Example 9

### Enzymatic Activity of Morpholino Deoxyribozyme Variants

The performance of the morpholino-based Dz858-15-15 variant is assessed in cell-free and intracellular RNA cleavage assays as described herein. These two assays were used successfully to screen our deoxyribozyme library and allow for a quick and easy determination of whether morpholinos are superior to phosphorothioate. Based on the literature, it is believed that morpholino-Dz858-15-15 may exceed the intracellular cleavage levels seen for Dz858-15-15end (i.e. <50%) and/or exhibit Km/Kcat values >5.6×10⁴ (mol/L)⁻¹ min⁻¹.

### Cell Free HCV RNA Cleavage Assay:

Deoxyribozyme cleavage efficiency, Km and Kcat values are evaluated for the phosphorothioate deoxyribozyme, Dz858-15-15end and the morpholino-Dz858-15-15 species. [³²P]-labeled HCV RNA spanning the HCV 5′UTR and adjoining core protein coding sequence are challenged with phosphorothioate- or morpholino-Dz858-15-15 and the degree of HCV RNA cleavage is evaluated in 6% polyacrylamide gels containing 8M urea as indicated herein. Results obtained for the unmodified and phosphorothioate-modified Dz858-15-15 and the morpholino-Dz858-15-15 are compared.

### Intracellular HCV RNA Cleavage Assay:

The morpholino-Dz858-15-15 and phosphorothioate-Dz858-15-15 are compared for their ability to cleave intracellular HCV RNA in liver cells as indicated herein. Briefly, plasmid, pHCV-UTR-core encoding the 942 base RNA segment from the HCV UTR and core protein coding sequence (HCV genome position 38 to 980) (21), is transfected into cells along with varying amounts of the tested variant. After 24 hours, polyA-RNA is isolated and HCV RNA cleavage quantified by RT-qPCR as indicated herein.

### Example 10

### In Vitro Toxicity of Variants

### In Vitro Toxicity Assays of Variants:

Phosphorothioate nucleotides may exhibit cytotoxic properties due to their affinity for cellular proteins. The toxicity of the morpholino-Dz858-15-15 and/or O-methyl-Dz858-15-15 variants is therefore evaluated in a cytotoxicity assay, metabolic assay and membrane integrity assay in comparison to the phosphorothioate-Dz858-15-15 and/or unmodified Dz-858-15-15.

### Cytotoxicity Assay:

Deoxyribozyme cytotoxicity is measured by exposing liver cells to increasing amounts of the phosphorothioate-Dz858-15-15, the morpholino-Dz858-15-15, the 2′ O-methyl-Dz858-15-15 and/or the unmodified Dz858-15-15 species. Conditions previously shown to give maximum intracellular HCV RNA cleavage±several log₁₀ doses are used. The number of viable cells are determined after 24 hours by differential acridine orange/ethidium bromide staining (Leeds, J. M., M. J. Graham, L. Truong, and L. L. Cummins. 1996. Anal. Biochem 235:36-43).

### Metabolic Assay:

Liver cells are treated with increasing concentrations of the phosphorothioate-Dz858-15-15, the morpholino-Dz858-15-15 deoxyribozyme species, the 2′ O-methyl-Dz858-15-15 or the unmodified Dz858-15-15 species. Following an additional 24 hours, cells are tested for loss in metabolic activity using the metabolic indicator MTS (Promega) and for changes in cell morphology.

### Membrane Integrity Assay:

Liver cells are tested for changes in cell membrane integrity using the lactate dehydrogenase (LDH) release assay CytoTox-ONE (Promega). When the deoxyribozyme promotes a loss of plasma membrane integrity, an increase in extracellular LDH is observed.

Cells are therefore treated for several hours with various concentrations of the phosphorothioate-Dz858-15-15, the morpholino-Dz858-15-15, the 2′ O-methyl-Dz858-15-15 or the unmodified Dz858-15-15 species or any other variant. The cells are washed and the medium is replaced with fresh medium for an additional 30-60 minutes. LDH levels are measured.

### Example 11

### In Vivo Studies of Variants

### Pharmacokinetics and Biodistribution

A single intravenous injection of saline or saline containing the various Dz858-15-15 (phosphorothioate-Dz858-15-15, the morpholino-Dz858-15-15, the 2′O-methyl-Dz858-15-15 and/or the unmodified Dz858) are injected into 5-7 week-old BALB/c mice (10/group). Pharmacokinetic and tissue distribution analyses are performed as described (Tanner, J. E. and A. Forte. 2002. abstr. AACR 93rd Annual Meeting, Orlando, Fla. 3195). Blood is collected at 5, 15, and 30 minutes and at 1, 2, 4, 8, 24, and 48 hours post-injection. The blood is clarified by centrifugation and stored at −80° C. until analysis. A second set of mice (5/group) are similarly injected and euthanized at 6, 12, 24 and 48 hours. Major vital organs are excised, rinsed in ice-cold saline and snap frozen on dry ice for storage at −80° C. Portions of liver from selected animals are treated with collagenase to release hepatocytes from nonparenchymal cells (i.e. Kupffer, endothelial, etc) (Nishikawa, M., S. Takemura, Y. Takakura, and M. Hashida. 1998. J. Pharmacol. Exp. Ther. 287:408-415). The amount of deoxyribozyme in these two liver cell types is measured to determine the relative distribution of deoxyribozyme in the liver.

Oligonucleotide biodistribution and pK from plasma and tissue samples for volumes less than 100 μl is performed using the Beckman P-ACE MDQ DNA system, column gel electrophoresis (CGE) apparatus. The various Dz858-15-15 (phosphorothioate-Dz858-15-15, the morpholino-Dz858-15-15, the 2′O-methyl-Dz858 and/or the unmodified Dz858-15-15) or their metabolic byproducts are extracted from plasma or tissue using a strong anion exchange extraction cartridge followed by desalting with reversed-phase C18 or phenyl-bonded cartridge (Yu, R. Z., et al., 2001, J. Pharm. Sci. 90:182-193). A final microdialysis step is performed on the sample prior to analysis in the Beckman P-ACE MDQ DNA system. The amount of Dz858-15-15 is measured using known quantities of Dz858-15-15 spiked in blank tissue (Leeds, J. M., et al. 1996. Anal. Biochem. 235:36-43). Pharmacokinetic variables are obtained using the Summit Research pK 2.0 software.

### In Vivo Toxicity Assays:

Based upon the pK findings, mice receive intravenous injections of saline or saline containing 5- and 50-fold deoxyribozyme doses (n=9/dose/3 sets) administered on days 1, 2, 7 and 15. Mice are observed daily and weighed. Three mice per group are sacrificed on days 3, 16 and 30, representing short, intermediate and long-term responses. At necropsy, a complete macroscopic evaluation of all body cavities is conducted, internal organs weighed and major body organs preserved in 10% neutral-buffered formalin for later histopathological evaluation by a contracted veterinary pathologist at Nucro-Technics (Scarborough, ON)(49)

### Example 12

### In Vivo Efficacy of Dz858

Genomic HCV was prepared as indicated in Example 6.

The human embryonic kidney cell line 293, obtained from the American Type Culture Collection (ATCC/CRL-1573), was cultured in Dulbecco's Modified Eagles Medium (DMEM) (Invitrogen Inc., Burlington, ON) supplemented with 10% fetal bovine serum (Medicorp Inc., Montreal, Qc) and antibiotics. The 293 cells were first seeded at 4.5×10⁵ cells per well in 6-well tissue culture plate and grown overnight. The 293 cells were then transfected in a final volume of 0.5 ml of Opti-MEM per well with 2.5 μg of the genomic-length HCV RNA using 7.5 μg of Lipofectamine 2000 (Invitrogen Inc.). After 5h, cells were removed from the plate using trypsin, washed in phosphate-buffered saline, and resuspended at a concentration of 1×10⁶ cells per 100 μl of matrigel (BD Biosciences, Mississauga, ON). 100 μl of Opti-MEM (Invitrogen) containing 7.5 μg of Lipofectamine 2000 or 7.5 μg Lipofectamine and 9 μg phosphorothioate-modified Dz858 were combined with the matrigel-293 mixture, respectively, immediately prior (less than 1 minute) to subcutaneous injection into the left or right flank of a mouse (n=9, strain Black-6 NOD/SCID/γ −/−). After 18 hours the matrigel plug containing HCV RNA-transfected 293 cells was recovered and total RNA was isolated using Trizol reagent (Invitrogen) and suspended in 40 μl of water.

HCV RT qPCR

The degree of in vivo HCV RNA cleavage was determined by reverse transcriptase quantitative polymerase chain reaction (RT-qPCR) analysis using the HCV primers described in Example 6.

The RT step was performed for 30 min followed by Taq activation by incubation at 95° C. for 10 min. PCR was performed by heating the sample for 10 min at 95° C., followed by 45 amplification cycles. Each amplification cycle consisted of a 15 sec incubation at 95° C. and a 1 min annealing and elongation step at 60° C. in a Mx3000P real-time PCR thermocycler (Stratagene Inc. La Jolla, Calif.). Logarithmic dilutions of pHCV-UTR-core containing HCV core sequences served as DNA reference standards during RT-qPCR analysis [21].

The level of cellular HCV RNA was normalized among each of the test tissue samples by measuring the level of RNA for the house-keeping gene glyceraldehyde-3-phosphate dehydrogenase (GAPDH) by performing RT-qPCR in conjunction with QuantiTect SYBR Green RT-PCR Kit (Qiagen) and human GAPDH sense primer: 5′-TCCCTCAAGATTGTCAGCAA-3′ (SEQ ID NO.:64) and antisense primer 5′-AGATCCACAACGGATACATT-3′ (SEQ ID NO.:65). The RT step was performed for 30 min followed by Taq activation by incubation at 95° C. for 10 min. PCR was performed for 45 amplification cycles. Each amplification cycle consisted of a 30-sec incubation at 95° C. and a 1-min annealing at 55° C. and elongation step at 72° C. for 30 sec in a Mx3000P real-time PCR thermocycler (Stratagene Inc. La Jolla, Calif.). Serial 2-fold dilutions of 293 cellular RNA served as reference standards during RT-qPCR analysis of GAPDH RNA.

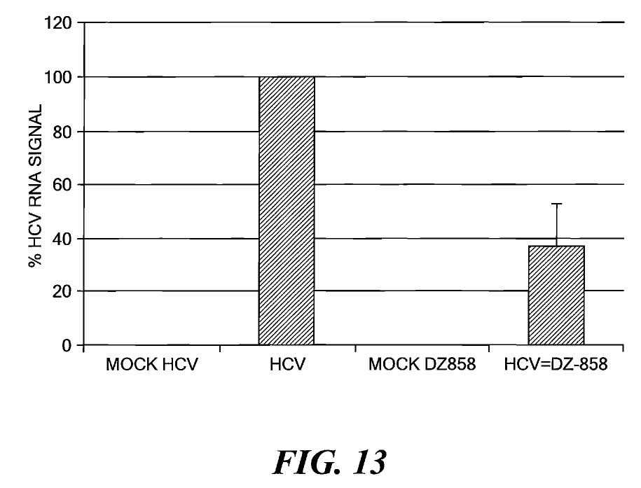

Results of this experiment shows a 63%±15.3 SEM reduction in HCV RNA levels in mice treated with phosphorothioate-modified Dz858 was observed (FIG. 13). Using the “one sample t-test” for the HCV versus HCV+Dz858 and a significance level of α=0.05, it was noted that the reduction of HCV RNA following Dz858 exposure was statistically significant (p-value=0.033).

Alternatively, although there is presently no small animal model which is universally accepted for HCV drug research (50), investigators have used murine models to gather useful preclinical information on the actions of deoxyribozymes (12). Liver cells stably transfected with pCMV-Dz858-target-GFPneo or pCMV-scrambled-Dz858-target-GFPneo are thus used as an alternative in vivo efficacy experimentation. Both of these transfectants express GFP, but the former mRNA is susceptible to cleavage by Dz858-15-15. Balb/C nu/nu mice (10/group) are injected i.p. with human liver cells stably expressing GFP from either pCMV-Dz858-target-GFPneo or pCMV-scrambled-Dz858-target-GFPneo. After 24 hours, the various Dz858-15-15 (phosphorothioate-Dz858-15-15, the morpholino-Dz858-15-15, the 2′O methyl-Dz858 and/or the unmodified Dz858-15-15) are injected into the tail vein. After an additional 24-48 hours, animals are euthanized and liver cells recovered by peritoneal lavage and Percoll banding. The liver cells are stained with primate-specific anti-human CD95 (PE-DX2, BD Pharmingen) (51), and differential expression of GFP protein following Dz858-15-15 treatment is determined by two-color FACS analysis. These tests allow us to gauge Dz858-15-15 in vivo efficacy.

The content of each publication, patent and patent application mentioned in the present application is incorporated herein by reference.

Although the present invention has been described in detail herein and illustrated in the accompanying drawings, it is to be understood that the invention is not limited to the embodiments described herein and that various changes and modifications may be effected without departing from the scope or spirit of the present invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### REFERENCES

1 Hoofnagle J H. Course and outcome of hepatitis C. Hepatology 2002; 36: S21-S29.
2 Shepherd J, Waugh N, Hewitson P. Combination therapy (interferon alfa and ribavirin) in the treatment of chronic hepatitis C: a rapid and systematic review. Health Technol Assess 2000; 4: 1-67.
3 Sookoian S C. New therapies on the horizon for hepatitis C. Ann Hepatol 2003; 2: 164-170.
4 Hugle T, Cerny A. Current therapy and new molecular approaches to antiviral treatment and prevention of hepatitis C. Rev Med Virol 2003; 13: 361-371.
5 Zein N N. Clinical significance of hepatitis C genotypes. Clin Microbiol Rev 2000; 13: 223-235.
6 Walker M P, Appleby T C, Zhong W, Lau J Y, Hong Z. Hepatitis C virus therapies: current treatments, targets and future perspectives. Antivir Chem Chemother 2003; 14: 1-21.
7 Oketani M, Asahina Y, Wu C H, Wu G Y. Inhibition of hepatitis C virus-directed gene expression by a DNA ribonuclease. J Hepatol 1999; 31: 628-634.
8 Bartolome J, Castillo I, Carreno V. Ribozymes as antiviral agents. Minerva Med 2004; 95: 11-24.
9 Shippy R, Lockner R, Farnsworth M, Hampel A. The hairpin ribozyme. Discovery, mechanism, and development for gene therapy. Mol Biotechnol 1999; 12: 117-129.
10 Opalinska J B, Gewirtz A M. Nucleic-acid therapeutics: basic principles and recent applications. Nat Rev Drug Discov 2002; 1: 503-514.
11 Kashani-Sabet M. Ribozyme therapeutics. J Investig Dermatol Symp Proc 2002; 7: 76-78.
12 Steele D, Kertsburg A, Soukup G A. Engineered catalytic RNA and DNA: new biochemical tools for drug discovery and design. Am J Pharmacogenomics 2003; 3: 131-144.
13 Goila R, Banerjea A C. Sequence specific cleavage of the HIV-1 coreceptor CCR5 gene by a hammer-head ribozyme and a DNA-enzyme: inhibition of the coreceptor function by DNA-enzyme. FEBS Lett 1998; 436: 233-238.
14 Kurreck J, Bieber B, Jahnel R, Erdmann V A. Comparative study of DNA enzymes and ribozymes against the same full-length messenger RNA of the vanilloid receptor subtype I. J Biol Chem 2002; 277: 7099-7107.
15 Santoro S W, Joyce G F. A general purpose RNA-cleaving DNA enzyme. Proc Natl Acad Sci USA 1997; 94: 4262-4266.
16 Wu Y, Yu L, McMahon R, Rossi J J, Forman S J, Snyder D S. Inhibition of bcr-abl oncogene expression by novel deoxyribozymes (DNAzymes). Hum Gene Ther 1999; 10: 2847-2857.
17 Yen L, Striftmatter S M, Kalb R G. Sequence-specific cleavage of Huntington mRNA by catalytic DNA. Ann Neurol 1999; 46: 366-373.
18 Chakraborti S, Banerjea A C. Inhibition of HIV-1 gene expression by novel DNA enzymes targeted to cleave HIV-1 TAR RNA: potential effectiveness against all HIV-1 isolates. Mol Ther 2003; 7: 817-826
19 Goila R, Banerjea A C. Inhibition of hepatitis B virus X gene expression by novel DNA enzymes. Biochem J 2001; 353: 701-708.
20 Takahashi H, Hamazaki H, Habu Y, et al. A new modified DNA enzyme that targets influenza virus A mRNA inhibits viral infection in cultured cells. FEBS Lett 2004; 560: 69-74.
21 Takamizawa A, Mori C, Fuke I et al. Structure and organization of the hepatitis C virus genome isolated from human carriers. J Virol 1991; 65: 1105-1113.
22 Mathews D H, Sabina J, Zuker M, Turner D H. Expanded sequence dependence of thermodynamic parameters improves prediction of RNA secondary structure. J Mol Biol 1999; 288: 911-940.
23 Zuker M. Mfold web server for nucleic acid folding and hybridization prediction. Nucleic Acids Res 2003; 31: 3406-3415.
24 Cairns M J, Hopkins T M, Witherington C, Sun L Q. The influence of arm length asymmetry and base substitution on the activity of the 10-23 DNA enzyme. Antisense Nucleic Acid Drug Dev 2000; 10: 323-332.
25 Sambrook J, Fritsch E F, Maniatis T. Mapping of RNA with ribonuclease and radiolabeled RNA probes. In: Nolan C, ed. Molecular cloning: A laboratory manual. 2^{nd} Ed. Plainview: Cold Spring Harbor Press, 1989: 7.71-7.78.
26 Breslauer K J, Frank R, Blocker H, Marky L A. Predicting DNA duplex stability from the base sequence. Proc Natl Acad Sci USA 1986; 83: 3746-3750.
27 Rychlik W, Spencer W J, Rhoads R E. Optimization of the annealing temperature for DNA amplification in vitro. Nucleic Acids Res 1990; 18: 6409-6412.
28 Santoro S W, Joyce G F. Mechanism and utility of an RNA-cleaving DNA enzyme. Biochemistry 1998; 37: 13330-13342.
29 Zhang X, Xu Y, Ling H, Hattori T. Inhibition of infection of incoming HIV-1 virus by RNA-cleaving DNA enzyme. FEBS Lett 1999; 458: 151-156.
30 Okamoto H, Okada S, Sugiyama Y et al. The 5′-terminal sequence of the hepatitis C virus genome. Jpn J Exp Med 1990; 60:167-177.
31 Cairns M J, Hopkins T M, Witherington C, Wang L, Sun L Q. Target site selection for an RNA-cleaving catalytic DNA. Nat Biotechnol 1999; 17: 480-486.
32 Scherr M, Rossi J J, Sczakiel G, Patzel V. RNA accessibility prediction: a theoretical approach is consistent with experimental studies in cell extracts. Nucleic Acids Res 2000; 28: 2455-2461.
33 Sioud M, Leirdal M. Design of nuclease resistant protein kinase c alpha DNA enzymes with potential therapeutic application. J Mol Biol 2000; 296: 937-947.
34 Asahina Y, Ito Y, Wu C H, Wu G Y. DNA ribonucleases that are active against intracellular hepatitis B viral RNA targets. Hepatology 1998; 28: 547-554.
35 Koff R S. Hepatitis vaccines. Infect Dis Clin North Am 2001; 15: 83-95.
36 Ferenci P. Predicting the therapeutic response in patients with chronic hepatitis C: the role of viral kinetic studies. J Antimicrob Chemother2004; 53: 15-18.
37 Locarnini S A. Mechanisms of drug resistance and novel approaches to therapy for chronic hepatitis C. J Gastroenterol Hepatol 2002; 17 Suppl 3: S351-S359.
38 Smith R M, Walton C M, Wu C H, Wu G Y. Secondary structure and hybridization accessibility of hepatitis C virus 3′-terminal sequences. J Virol 2002; 76: 9563-9574.
39 Richardson P, Kren B T, Steer C J. In vivo application of non-viral vectors to the liver. J Drug Target 2002; 10: 123-131.
40 Wattiaux R, Jadot M, Laurent N, Dubois F, Wattiaux-De Coninck S. Cationic lipids delay the transfer of plasmid DNA to lysosomes. Biochem Biophys Res Commun 1996; 227: 448-454.
41 Stein C A. Antitumor effects of antisense phosphorothioate c-myc oligodeoxynucleotides: a question of mechanism. J Natl Cancer Inst 1996; 88: 391-393.
42 Kurreck J. Antisense technologies. Improvement through novel chemical modifications. Eur J Biochem 2003; 270: 1628-1644.
43 Juliano R L, Yoo H. Aspects of the transport and delivery of antisense oligonucleotides. Curr Opin Mol Ther 2000; 2: 297-303.
44 Khachigian L M. Deoxyribozymes: cutting a path to a new class of therapeutics. Curr Opin Mol Ther 2000; 4:119-121.
45 Santiago F S, Lowe H C, Kavurma M M, Chesterman C N, Baker, Atkins D G, Khachigian L M. New DNA enzyme targeting Egr-1 mRNA inhibits vascular smooth muscle proliferation and regrowth after injury. Nat Med 1999; 5: 1264-1269.
46 Santiago F S, Khachigian L M. Nucleic acid based strategies as potential therapeutic tools: mechanistic considerations and implications to restenosis. J Mol Med 2002; 79: 695-706.
47 Trepanier et al. 2006. J Viral Hepat 13:131-138
48 Schubert, S and J. Kurreck 2004 Curr Drug Targets 5:667-681
49 Tanner, J. E., et al., 2004. Mol. Cancer Res. 2:281-288)
50 Pietschmann, T. and R. Bartenschlager. 2003. Clin. Liver Dis. 7:23-43
51 Marusawa, H., et al., 2001. Microbiol. Immunol. 45:483-489

1. A deoxyribozyme comprising a first and second annealing arm substantially complementary to a target HCV core region, said deoxyribozyme comprising a catalytic region able to cleave said target HCV core region between said first and second annealing arm.

2. The deoxyribozyme of claim 1, wherein said target HCV core region is substantially conserved among HCV subtypes.

3. The deoxyribozyme of claim 1, wherein said target HCV core region is accessible for annealing with said deoxyribozyme.

4. The deoxyribozyme of claim 1, wherein said first and second annealing arm each independently has from about 7 to 20 deoxyribonucleotides and wherein said deoxyribozyme binds a HCV region located between nucleotide 330 and nucleotide 370 of HCV sequence depicted in SEQ ID NO.:1, a HCV region located between nucleotide 676 and nucleotide 715 of HCV sequence depicted in SEQ ID NO.:1 or a HCV region located between nucleotide 835 and nucleotide 880 of HCV sequence depicted in SEQ ID NO.:1

5. The deoxyribozyme of claim 4, wherein said deoxyribozyme is able to cleave said HCV region at a site defined by 5′-A₁-R/Y-A₂-3′, wherein A₁ is a first annealing region of about 7 to 20 nucleotides, A₂ is a second annealing region of about 7 to 20 nucleotides, wherein R is A or G and wherein Y is U or C.

6. The deoxyribozyme of claim 5, wherein R is A and Y is U or C.

7. The deoxyribozyme of claim 4, wherein said first and second annealing arm each independently has from about 7 to 18 deoxyribonucleotides.

8. The deoxyribozyme of claim 4, wherein said first and second annealing arm each independently has from about 9 to 15 deoxyribonucleotides.

9. The deoxyribozyme of claim 4, wherein said first and second annealing arms are totally complementary to said HCV region.

10. The deoxyribozyme of claim 4, wherein said first or second annealing arms possess one nucleotide which is not complementary to said HCV region.

11. The deoxyribozyme of claim 1, wherein said deoxyribozyme is capable of intracellular cleavage of a HCV sequence.

12. The deoxyribozyme of claim 11, wherein said HCV sequence is a HCV genome or a portion thereof.

13. The deoxyribozyme of claim 1, wherein said deoxyribozyme is capable of cleaving a HCV sequence found in a mammal.

14. The deoxyribozyme of claim 13, wherein said HCV sequence is a HCV genome or a portion thereof.

15. The deoxyribozyme of claim 1, wherein said deoxyribozyme is from about 25 to about 55 deoxyribonucleotides long.

16. The deoxyribozyme of claim 1, wherein said deoxyribozyme comprises at least one phosphorothioate-derivative nucleotide, at least one 2′-O-methyl nucleotide analog or at least one morpholino-derivative nucleotide.

17. The deoxyribozyme of claim 16, wherein said derivative or analog is located at one or both ends of said deoxyribozyme.

18. The deoxyribozyme of claim 16, wherein said derivative or analog is located within said first and/or second arm.

19. The deoxyribozyme of claim 1, wherein said target HCV core region is a messenger RNA or a genomic RNA.

20. The deoxyribozyme of claim 1, wherein said target HCV core region is single stranded.

21. The deoxyribozyme of claim 1, wherein said catalytic region comprises a type I domain or a type II domain or a variant thereof.

22. The deoxyribozyme of claim 1, whereby upon hybridization of said deoxyribozyme and target to form a complex, said complex comprises an unpaired purine followed by a paired pyrimidine located at the junction between said first and second annealing arms.

23. A deoxyribozyme able to cleave a target HCV core region intracellularly, said deoxyribozyme comprising formula X₁-Cₐ-X₂, wherein X₁ is a first annealing arm having a nucleotide sequence of from 7 to 20 deoxyribonucleotides, Cₐ is a type I or type II catalytic domain and X₂ is a second annealing arm having a nucleotide sequence of from 7 to 20 deoxyribonucleotides wherein said deoxyribozyme is substantially complementary to a HCV sequence located between nucleotides 330 and 370 of SEQ ID NO.:1, or between nucleotides 676 and 715 of SEQ ID NO.:1, or between nucleotide 835 and 880 of SEQ ID NO.:1.

24. A composition comprising;
a. at least one deoxyribozyme of claim 1, and
b. a pharmaceutically acceptable carrier.

25. A method of treating a mammal having or susceptible of having a HCV infection, the method comprising administering the composition of claim 24 to said mammal.

26. A method of generating a deoxyribozyme, the method comprising allowing synthesis of a deoxyribozyme comprising formula X₁-Cₐ-X₂, wherein X₁ is a first annealing arm having a nucleotide sequence of from 7 to 20 deoxyribonucleotides, Cₐ is a type I or type II catalytic domain and X₂ is a second annealing arm having a nucleotide sequence of from 7 to 20 deoxyribonucleotides wherein said deoxyribozyme is substantially complementary to a HCV sequence located between nucleotides 330 and 370 of SEQ ID NO.:1, or between nucleotides 676 and 715 of SEQ ID NO.:1, or between nucleotide 835 and 880 of SEQ ID NO.:1.

27. The method of claim 26, wherein said synthesis is done chemically and wherein said deoxyribonucleotides comprises at least one modified nucleotide or at least one deoxyribonucleotide is replaced with a nucleotide analog.
